(19) United States
(12) Patent Application Publication
(10) Pub. No.: US 2025/0166765 A1
(43) Pub. Date: May 22, 2025

(54) Multi-Agent Bioinformatics System for Single Cell Data Analytics

(71) Applicant: TERRAFLOW BIOINFORMATICS CORP., Cambridge, MA (US)

(72) Inventors: Daniel Freeman, Cambridge, MA (US); Pratip Chattopadhyay, Cambridge, MA (US)

(21) Appl. No.: 18/953,018

(22) Filed: Nov. 19, 2024

Related U.S. Application Data
(60) Provisional application No. 63/601,129, filed on Nov. 20, 2023.

(51) Int. Cl. G16H 15/00; G16B 25/10; G16B 50/30; G16H 50/70; G16H 70/00

(52) U.S. Cl. CPC G16H 15/00; G16B 25/10; G16B 50/30; G16H 50/70; G16H 70/00

(57) ABSTRACT

A multi-agent system for transforming single call data insights about phenotypes exhibited by the single cell data can comprise one or more processors and databases, the database(s) configured to provide linkages between (1) phenotype data for phenotypes derived from the single cell data and (2) external knowledge about the phenotypes derived from medical literature. The system can include first, second, and third agents configured for operating in parallel with each other; the first agent(s) configured to transform the single cell data into the phenotype data; the second agent(s) configured to process a medical literature corpus using NLP to determine information relating to the phenotypes described by the medical literature, wherein the external knowledge comprises the determined information; and the third agent(s) configured to synthesize insight(s) about the single cell data with respect to one or more phenotypes of interest, wherein the insight(s) are derived from the linkages in the database(s).

### CROSS-REFERENCE AND PRIORITY CLAIM TO RELATED PATENT APPLICATION

This patent application claims priority to U.S. provisional patent application 63/601,129, filed Nov. 20, 2023, and entitled “Systems and Methods for Single Cell Data Analytics”, the entire disclosure of which is incorporated herein by reference.

### INTRODUCTION

The proteins associated with cells provide critical information about nearly all aspects of their life cycle, including lineage, maturation, distribution, proliferation, and function. Biomedicine technologies—such as flow cytometry, and other emerging single-cell platforms—are used to quantify and describe cells according to the specific combinations of proteins they express. From this information, researchers can glean important insights into the fundamentals of cell biology. For example, most of our understanding of how immune responses work (and how immune therapies generate effective responses) comes from these kinds of measurements.

In disease and treatment settings, the measurement of cellular proteins allows us to quantify the number of a particular cell type in an individual and compare those frequencies across patient groups. When a cell type is significantly elevated or diminished in a group of patients, we infer that this cell type is a biomarker that may predict some aspect of disease or treatment.

Biomarker discovery and classification is a difficult technical challenge in biomedical research. A classical method defines immune cell subsets by the combination of proteins they express or lack on their surface. For example, naïve T cells are often defined as CD3+CD45RA+CCR7+CD57−. Of these, CD3 is a universal marker of T cells, and the particular combination of CD45RA and CCR7 expression, combined with the lack of CD57 expression, identifies these cells as naïve. Accordingly, it should be understood that each additional marker in a combination of protein markers resolves subtypes and substrates with deeper levels of granularity. There are hundreds of such combinations associated with specific cell types in the immune system.

Scientists traditionally hand-gate populations of interest using interactive software like FlowJo. Manual gating (e.g., see FIG. 1A which shows an example of manual analysis where users hand-draw gates around cell types of interest) is a time-consuming and unreliable process. Scientists have to know what cell types to measure ahead of time, which many do not. Even if they do manage to measure all the major immune populations, they may be missing other cell types that are poorly understood but still important to their study. They never know if there's another cell type out there that perfectly captures the phenotype they're looking for.

Conventional machine learning (ML) approaches offer an unbiased approach to biomarker discovery, but suffer from producing results that are ambiguous (see FIG. 1B-which shows examples where users get back multicolored Uniform Manifold Approximation and Projection (UMAP) plots or heatmaps and have to figure out what the cell types actually mean).

For example, FlowSOM (see Quintelier, Katrien, et al. “Analyzing high-dimensional cytometry data using FlowSOM, Nature Protocols, 16, 3775-3801 (2021)) groups cells together based on similarities in protein expression and checks if any clusters correlate with treatment outcome.

As another example, CellCNN (see “Arvaniti, E. and Claassen, M., “Sensitive detection of rare diseases-associated cell subsets via representation learning”, Nature Communications, 8, Article number: 14825 (2017)) trains a neural network to predict patient labels and uses filter weights to represent cell types.

Machine learning models can be difficult to interpret and validate. Cell types are based on complex transformations of all 30 protein markers, forcing scientists to use abstract weights and visualizations to understand what the cell types actually mean. They have to use fully-trained machine learning models every time they want to measure the target population, which often fail to adapt to day-to-day changes in instrument, antibody, or protocol, much less new institutional settings. Moreover, scientists can't replicate results on lower-parameter instruments typically used in clinical settings.

As such, there are many technical shortcomings with existing techniques for analyzing single cell data sets. Disclosed herein are techniques that provide technical solutions to one or more of these technical shortcomings in the art by providing for single cell data analytics that combine single cell data with techniques for determining insights about the single cell data based on transformations of the single cell data. For example, the transformed single cell data can be combined with practical applications of computerized natural language processing (NLP) and/or natural language generation (NLG) to generate insights about the single cell data that are augmented by relevant medical literature.

For example, disclosed herein are techniques for transforming single cell data (where the single cell data comprises measurements indicative of whether various protein markers are present in the single cells of samples) into a network model of related phenotypes. The network model can serve as a logical model of the single cell data that comprises a plurality of nodes which correspond to phenotypes derived from the single cell data, where the phenotypes comprise combinations of one or more protein markers from the single cell data. Each node of the network model can be associated with relationship data that indicates a relationship between the node's corresponding phenotype and an outcome being evaluated (e.g., a disease status, treatment status, etc.) as indicated by the single cell data. The nodes of the network model are connected with each other according to a network structure that is based on the relatedness of the nodes' corresponding phenotypes.

The network model of related phenotypes can be linked to phenotype-relevant medical literature using a data structure that associates a plurality of medical papers with the phenotypes that are referenced in those medical papers. The network model and the linked phenotype-relevant medical papers can then be leveraged using analytical techniques to determine insights about phenotypes of interest for presentation to users.

The network structure of the logical model that defines the network model of related phenotypes interconnects the nodes of the network model as a function of degrees of relatedness between the nodes' corresponding phenotypes. Thus, the higher the similarity between two phenotypes in terms of their protein marker compositions, the fewer the number of connections between the nodes of those two phenotypes in the network model. As an example, where two phenotypes differ only by the addition or removal of a single protein marker, the nodes for those two phenotypes can be directly connected with each other in the network model (e.g., a single connection hop would be needed to traverse the network model from one of those phenotypes to the other). By contrast, where two phenotypes differ by the addition or removal of two or more protein markers, the nodes for those two phenotypes would be indirectly connected with each other in the network model (e.g., hopping from one phenotype to the other would need to traverse through one or more intervening nodes of the network model). The imposition of this network structure on the single cell data stands in stark contrast to the flat nature of the raw single cell data which effectively lists measurement intensities for different protein markers with respect to different single cells of a sample. Performing analytics on this flat single cell data is computationally daunting and will often provide users with results that are not contextually cohesive. By contrast, the network structure that is imposed on the single cell data by the techniques described herein makes the data structure that stores the single cell data in accordance with the network model of related phenotypes more amenable to computerized analysis to discover insights that would otherwise be hidden because the network model will self-describe the relatedness of the phenotypes derived from the single cell data so that the transformed single cell data can be efficiently analyzed in the context of similar phenotypes.

While example embodiments are described below where the data comprises single cell protein measurements from multiple samples, including flow cytometry and mass cytometry data, it should be understood that the techniques described herein can also be used for other types of single cell data, including but not limited to single cell data for transcript expression (RNA-seq), metabolite expression (mass spectrometry), chromatin accessibility (ATAC-seq), and multiomic techniques such as CITE-seq that combine two or more omic data types. In general, it is expected that the techniques described herein can be applied to any situations where a user wants to define cell types in a single cell experiment using simple combinations of molecular features where such features can be mapped to a standardized database of molecule IDs.

In view of the foregoing, disclosed herein are several innovative techniques for improving single cell data analytics.

According to a first example, disclosed herein are techniques for automating a transformation of single call data into one or more insights about the single cell data with respect to one or more phenotypes exhibited by the single cell data. The single cell data can comprise data indicative of protein expression levels for a plurality of different protein markers in a plurality of different single cells with respect to a plurality of subjects, and wherein each subject is associated with status data for the defined outcome. According to an example, one or more processors can be configured to carry out operations that include (1) translating the single cell data into a network model of related phenotypes, wherein each phenotype represents a protein marker composition of one or more of the different protein markers from the single cell data, the network model comprising a plurality of nodes that are connected in a network structure, each node corresponding to a different phenotype from among a plurality of the phenotypes and being associated with relationship data for its corresponding phenotype with respect to the defined outcome, and wherein the network structure is derived from relatedness between the protein marker compositions of the nodes' corresponding phenotypes; (2) linking one or more phenotypes from the network model with one or more medical papers that describe the one or more phenotypes based on a data structure that associates a plurality of medical papers with phenotypes that are described in the medical papers; and (3) determining one or more insights about the single cell data with respect to one or more phenotypes of interest based on (i) the network structure of the network model, (ii) the relationship data, and (iii) one or more medical papers that are linked to the one or more phenotypes of interest. The network structure for the network model provides efficient computerized processing of the network model, which facilitates making connections with medical literature that contextualize insights about the single cell data with respect to phenotypes of interest and other closely related phenotypes according to the network model. As an example, the network structure can define direct connections within the network model between nodes where the nodes' corresponding phenotypes differ by the addition or removal of 1 protein marker.

As another example, disclosed herein is a system for transformation of single cell data, the system comprising one or more memories and one or more processors for cooperation with the one or more memories. The one or more memories can be configured to store a data structure, wherein the data structure comprises a network model of related phenotypes, wherein the network model comprises a plurality of nodes that are connected with each other according to a network structure, wherein the nodes correspond to different phenotypes derived from the single cell data so that each node corresponds to a different phenotype among the phenotypes, wherein each node is associated with relationship data indicative of a relationship between the node's corresponding phenotype and a defined outcome according to the single cell data, wherein each phenotype represents a protein marker composition of one or more of the different protein markers from the single cell data, and wherein the network structure defines connections within the network model between nodes where the nodes' corresponding phenotypes differ by the addition or removal of 1 protein marker. The one or more processors can be configured to (1) access and process the data structure to identify one or more phenotypes of interest based on (i) the network structure of the network model and (ii) the relationship data, (2) connect to a natural language generation (NLG) platform via an application programming interface (API), (3) provide input to the NLG platform via the API to trigger the NLG platform to generate one or more insights about the single cell data with respect to the identified one or more phenotypes of interest, (4) receive the generated insight(s) from the NLG platform, and (5) generate a natural language output based on the received generated insight(s).

Further still, according to another example, disclosed herein is a multi-agent system for transforming single call data into one or more insights about one or more phenotypes exhibited by the single cell data, where the system comprises (1) one or more processors, (2) one or more databases, (3) one or more first agents, (4) one or more second agents, and (5) one or more third agents. The one or more databases can be configured to provide a plurality of linkages between (1) phenotype data for a plurality of phenotypes derived from the single cell data and (2) external knowledge about the phenotypes derived from medical literature. The one or more first agents are for execution by the one or more processors and can be configured to transform the single cell data into the phenotype data. The one or more second agents are for execution by the one or more processors and can be configured to process a corpus of medical literature using natural language processing (NLP) to determine information relating to the phenotypes described by the medical literature, wherein the external knowledge comprises the determined information. The one or more third agents are for execution by the one of more processors and can be configured to synthesize one or more insights about the single cell data with respect to one or more phenotypes of interest, wherein the one or more insights are derived from the linkages in the database. Moreover, the first, second, and third agents can be configured for operating in parallel with each other.

The phenotype data created by the one more first agents can comprise a network model of related phenotypes derived from the single cell data, where the network model exhibits a network structure that connects the phenotypes as a function of their degrees of relatedness to each other. Further still, the one or more second agents may be configured to augment the network model of related phenotypes with external knowledge derived from medical literature and/or a medical ontology that is found to be relevant to the phenotypes.

According to another example, disclosed herein are embodiments for a synthesis agent that can be executed to determine one or more insights relating to single cell data. For example, an article of manufacture is disclosed that comprises processor-executable code resident on a non-transitory computer-readable storage medium, wherein the processor-executable code is configured for execution by one or more processors to cause the one or more processors to (1) traverse a network model of related phenotypes that is derived from single cell data to identify one or more phenotypes of interest, wherein the network model comprises a plurality of nodes that are connected according to a network structure, wherein the nodes correspond to different phenotypes derived from the single cell data and are associated with relationship data between the nodes' corresponding phenotypes and a defined outcome according to the single cell data, wherein the phenotypes are defined by protein marker combinations of one or more protein markers from the single cell data, wherein the network structure defines connections within the network model between nodes where the nodes' corresponding phenotypes differ by the addition or removal of 1 protein marker, and wherein the one or more phenotypes of interest are identified based on (i) the network structure and (ii) the relationship data, (2) determine medical literature metadata that is linked to the identified one or more phenotypes of interest and has been derived from a plurality of phenotype-related medical papers, and (3) synthesize an insight about the single cell data with respect to the identified one or more phenotypes of interest based on the determined medical literature metadata.

According to another example, disclosed herein are techniques for linking single cell data with a data structure of medical knowledge external to the single cell data. The single cell data can comprise data indicative of protein expression levels for a plurality of different protein markers in a plurality of different single cells with respect to a plurality of subjects, and wherein each subject is associated with status data for the defined outcome. One or more processors can perform operations that include (1) translating the single cell data into a network model of related phenotypes, wherein each phenotype represents a protein marker composition of one or more of the different protein markers from the single cell data, the network model comprising a plurality of nodes that are connected in a network structure, each node corresponding to a different phenotype from among a plurality of the phenotypes and being associated with relationship data for its corresponding phenotype with respect to the defined outcome, and wherein the network structure defines connections within the network model between nodes where the nodes' corresponding phenotypes differ by the addition or removal of 1 protein marker; (2) accessing the data structure of medical knowledge, wherein the data structure of medical knowledge associates a plurality of different items of medical knowledge with phenotypes relevant to the medical knowledge items; and (3) linking a plurality of the nodes of the network model with medical knowledge items based on the accessed data structure according to a correspondence between the phenotypes corresponding to the linked nodes and the phenotypes associated by the data structure of medical knowledge with medical knowledge items. The data structure of medical knowledge may comprise an index of phenotype-related medical papers and/or an ontology of medical terms.

Further still, disclosed herein are examples of techniques for processing a corpus of medical literature that comprises a plurality of medical papers to create a computer-readable data structure using natural language processing (NLP) so that the computer-readable data structure associates the papers with (1) a plurality of phenotypes described in the papers as determined via NLP and (2) metadata about the papers. One or more processors can perform operations that include (1) searching machine-readable text of the papers based on a plurality of keywords that are indicative of phenotype delimiters in the text; (2) identifying a plurality of phenotype-related papers based on the searching; (3) extracting phenotypes from the identified phenotype-related papers using NLP; (4) determining metadata for the identified phenotype-related papers; and (5) creating a computer-readable data structure that indexes the phenotype-related papers by identifiers for the phenotypes extracted therefrom and associates the phenotype-related papers with their determined metadata.

As yet another example, disclosed herein is a technically innovative approach for processing single cell data to evaluate whether a plurality of protein markers are expressed by the single cell data. The single cell data can comprise a plurality of fluorescent intensity values for a plurality of protein markers with respect to a plurality of cells. One or more processors can perform operations that include (1) normalizing the fluorescent intensity values by linearizing the fluorescent intensity values using a logical function and rescaling the linearized fluorescent intensity values; (2) calibrating a sigmoidal function by defining an inflection point for the sigmoidal function so that the calibrated sigmoidal function inflates values for high-expressing cells over neutral and low-expressing cells; (3) transforming the normalized fluorescent intensity values using the calibrated sigmoidal function; (4) defining a plurality of phenotypes that comprise protein marker combinations of one or more protein markers from the single cell data; (5) weighing the phenotypes by computing root products of the transformed normalized fluorescent intensity values of the phenotypes' component protein markers; (6) computing average cell weights within a plurality of samples of the single cell data based on the computed root products; and (7) using the computed average cell weights as indicators of phenotype expression by the cells of the single cell data. These non-gated indicators of protein expression can be used to create the network model of related phenotypes from single cell data as described herein.

Also disclosed herein is a system for processing single cell data, where the system comprises (1) one or more processors configured to transform single cell data into a set of linkages between (i) a plurality of phenotypes derived from the single cell data and (ii) external medical knowledge, wherein the external medical knowledge is derived from medical literature and/or a medical ontology, and (2) one or more databases configured to store the linkages. In an example embodiment, this transformation can include a creation of a network model of related phenotypes from the single cell data, where the network model exhibits a network structure that connects phenotypes as a function of their degrees of relatedness to each other. Moreover, the linkages produced by this transformation can be analyzed to produce one or more insights about phenotypes of interest, which may include insights expressed in natural language that can be produced by generative artificial intelligence (AI) models such as generative large language models (LLMs).

Further still, disclosed herein are techniques where computational latency can be greatly reduced via an innovative applications of parallelized compute resources. For example, various processing operations involving matrix manipulations can be offloaded from one or more CPUs for the system to one or more parallelized compute resources such as one or more graphics processing units (GPUs) that can serve as offload engines for the CPU(s).

As an example, disclosed herein is a method for transformation of single cell data from a first model to a second model, the method comprising (1) combining a first matrix with a second matrix to compute a third matrix, wherein the first matrix and the second matrix serve as the first model of the single cell data, wherein the first matrix comprises a cell-by-protein marker matrix of data indicative of expressions of a plurality of protein markers in a plurality of cells of a patient, wherein the second matrix comprises a phenotype-by-protein marker matrix of data indicative of which of the protein markers are present in a plurality of phenotypes, and wherein the third matrix comprises a cell-by-phenotype matrix of data indicative of which of the phenotypes are present in which of the cells according to the first and second matrices, and (2) aggregating data within the third matrix to generate a fourth matrix, wherein the fourth matrix comprises data indicative of aggregated phenotype abundance in the cells, wherein the fourth matrix serves as the second model of the single cell data, where the combining and aggregating steps are carried out by one or more parallelized compute resources that perform a plurality of arithmetic operations for the combining and aggregating steps in parallel. A corresponding system for this transformation method is also disclosed.

As another example, also disclosed herein is a method for transforming single cell data from a first model to a second model, wherein the first model comprises a first matrix, wherein the first matrix is a cell-by-phenotype matrix of data indicative of which of a plurality of phenotypes are present in which of a plurality of cells of the single cell data, wherein each phenotype comprises a combination of one or more of a plurality of different protein markers from the single cell data, the method comprising (1) encoding the phenotypes using a plurality of prime number marker factors so that the phenotypes are represented by unique numerical encodings, wherein the prime number marker factors are assigned to the protein markers so that each unique numerical encoding for a subject phenotype comprises a product of the assigned prime number marker factors for each constituent protein marker of the subject phenotype, and (2) identifying which of the phenotypes qualify as rare phenotypes according to defined criteria using prime factorization derived from the encoding of the phenotypes, wherein the rare phenotypes are excluded from computing values that populate a second matrix that serves as the second model, and wherein the second matrix comprises values indicative of aggregated phenotype abundance in the cells, and where the identifying step is performed by one or more parallelized compute resources that perform a plurality of arithmetic operations for the identifying step in parallel. A corresponding system for this transformation method is also disclosed.

These innovative techniques provide a number of technical advantages over conventional computerized solutions in the art, where these technical advantages include:
Speed, memory, and security. Mining an entire literature database every time a user uploads a dataset to a computer system would be computationally infeasible. Moreover, it would require making public API calls that could expose sensitive information about the experiment. With the example approaches described herein, relevant literature can be converted into a linked database that we can easily store and query against new datasets. Moreover, because of the network structure of the network model of related phenotypes, phenotypes of interest can be efficiently searched and interpreted in the context of similar phenotypes so that assessments can be made regarding where noteworthy insights can be found.
Interpretability. Existing techniques rely on visual inspection of abstract representations, making it difficult to understand what results actually mean. For example, FlowSOM splits cells into a predefined number of clusters and reports mean protein expression within each cluster. As shown by FIG. 1C, we can see that MetaClust3 correlates with disease status and has elevated levels of HLADR. However, it's not clear if HLADR expression is sufficient to define this population or if you actually need two, four, or all twenty proteins to truly recapitulate that phenotype. As such, FlowSOM is not clear about which proteins are necessary to define each population. You never really know what a conventional machine learning model is looking at so you have to rely on post-hoc feature important analyses. By contrast, the innovative techniques disclosed herein can operate to define each population with a simple phenotype and an explicit gating path that can be directly interpreted and compared to existing literature (which can explain why the phenotype matters biologically) and that users can replicate and validate on their own.
Depth of analysis/Completeness. Machine learning algorithms may only report a subset of cell types affected by disease. For example, CellCNN learns a set of filters that helps the model classify patients. Each filter represents a unique disease-associated cell type, and larger weights correspond to more influential proteins. Thus, CellCNN uses learned filter weights to represent disease-associated cell types, and the CellCNN classifier may only report a subset of cell types affected by disease. In the example shown by FIG. 1D, we can see that two filters are sufficient to classify healthy and diseased samples with 100% accuracy. However, there may be other cell types in the data that are biologically meaningful but not necessary for the prediction task. Traditional algorithms are not “incentivized” to learn populations beyond those needed to generate accurate predictions. By contrast, the innovative techniques described herein can perform an exhaustive search for disease-associated phenotypes and operate to consider every combination of proteins that a scientist could possibly measure with their data. The combinatoric network approach as discussed herein gives scientists a total view of their data, allowing them to draw connections they would've missed using existing methods.
Orthogonal validation. The biomedical world is facing a crisis in non-reproducible data. This is especially true in the flow cytometry world, where scientists often have to draw insights from a few precious samples. The innovative technology disclosed herein can de-risk biomarkers by prioritizing phenotypes that have already been reported in similar settings.
Ease of use. Existing techniques require users to anticipate the number of clusters or tune arcane machine learning parameters. The wrong parameters can produce misleading or inaccurate results, yet most biologists do not understand how to select and evaluate models appropriately. Because the innovative techniques disclosed herein can be based on simple rules (e.g., does adding a marker improve correlation or not?), we don't need to adjust the algorithm on each new dataset. Users can simply provide the system with raw data and patient labels; and they can receive a complete immunophenotyping report (e.g., in a report sent to their inbox or otherwise presented to the user).
Replicability. Machine learning algorithms are notoriously sensitive to batch effects, especially in flow cytometry. Changes in instrument voltage, antibody lot, sample handling, etc. change results from day to day, let alone institute to institute. A change in just one protein marker can throw off the whole model. The innovative techniques disclosed herein sidestep this issue through the context-relevant network modeling and the simplicity by which phenotypes are formed and evaluated. Even a junior cytometrist could gate GITR+PD1⁺ T-cells and see that they do indeed correlate with outcome—they don't need a black-box model to do it for them.
Scalability. Traditional algorithms define populations with complex transformations of the entire input dataset. If a user trains a model with a 30-marker panel, they better make sure they use the same 30 antibodies on every subsequent experiment. By contrast, the innovative techniques disclosed herein can be used to select populations that can be gated with just 1-2 markers, only going up to three or more if necessary to define the target populations. This makes it easy to validate populations on lower-parameter instruments typically used in clinical settings.
Generalizability. Machine learning models tend to overfit small datasets, hyperfocusing on random artifacts that do not generalize to new data. The innovative techniques disclosed herein can be used to mitigate overfitting by prioritizing phenotypes that have already been validated in the literature. As such, the technology disclosed herein helps scientists build on existing biomedical knowledge and maximize translational success.

These and other features and advantages are described in greater detail below.

### BRIEF DESCRIPTION OF THE DRAWINGS

FIGS. 1A-1D show examples of conventional single cell analytics techniques in the art.

FIG. 2 shows an example process flow for transforming single cell data into one or more insights about the single cell data.

FIG. 3 shows an example set of single cell data.

FIG. 4 shows an example network model of related phenotypes.

FIGS. 5A-5C show example techniques for identifying phenotypes of interest.

FIG. 6 shows an example process flow for linking phenotypes to relevant medical literature and determining insights about single cell data with respect to phenotypes of interest.

FIG. 7A shows an example prompt template that can be used in an example embodiment.

FIG. 7B shows an example prompt that can be provided to an NLG platform.

FIG. 7C shows another example prompt that can be provided to an NLG platform.

FIG. 7D shows an example NLG output that can be generated from the prompt of FIG. 7C using techniques described herein.

FIGS. 7E and 7F show an example API specification for interfacing with an NLG platform.

FIG. 8A shows an example process flow for mapping medical literature metadata onto a network model.

FIG. 8B shows an example of a network model of related phenotypes that has been augmented with mapped medical literature metadata.

FIG. 8C shows another example process flow using linkages between phenotypes and relevant medical literature to determine insights about single cell data with respect to phenotypes of interest.

FIG. 9A shows an example process flow for translating single cell data into a network model of related phenotypes.

FIG. 9B shows an example combinatoric matrix that identifies the measured phenotype abundance for each phenotype with respect to each patient of the single cell data.

FIG. 9C shows an example of how relationship data values for phenotypes can be computed from single cell data.

FIG. 9D shows another example of a network model of related phenotypes that can be generated and displayed using the techniques of FIG. 9A.

FIG. 9E shows an example architecture that offloads various data processing operations to a graphics processing unit (GPU).

FIG. 9F shows an example process flow that can be carried out by the GPU of FIG. 9E to implement a rare phenotype exclusion filter.

FIG. 10 shows an example bioinformatics system or platform that can be configured to carry out the transformation of single cell data into insights about the single cell data using techniques as discussed herein.

FIG. 11 shows another example process flow for transforming single cell data into insights about the single cell data using techniques disclosed herein.

FIG. 12 shows an example network model of related phenotypes that has been augmented with medical literature metadata.

FIG. 13 shows an example of a natural language insight that can be produced from single cell data using the techniques described herein.

FIG. 14 shows an example process flow for creating a literature index of phenotype-related medical papers.

FIG. 15 shows another example process flow for creating a literature index of phenotype-related medical papers.

FIG. 16 shows an example of a JSON string for an extracted phenotype and associated metadata.

FIG. 17 shows an example of how unstructured text can be translated into an extracted set of protein markers (which define a phenotype) and then further translated into a database entry in a literature index.

FIG. 18 shows another example bioinformatics system or platform that can be configured to carry out the transformation of single cell data into insights about the single cell data using techniques as discussed herein.

FIG. 19 shows an example technique for computing specificity scores for medical papers with respect to a phenotype.

FIG. 20 shows an example for computing a phenotype literature score.

FIG. 21 shows an example of how scoring can link phenotypes with relevant medical literature and disease classifications.

FIG. 22 shows another example process for determining insights from single cell data.

FIG. 23 shows another example of a network model of related phenotypes that has been integrated with a medical ontology.

FIG. 24 shows an example of how the system can be used to annotate phenotype nodes with an appropriate level of specificity.

FIG. 25 shows an example of database linkages that can be created between phenotypes, medical papers, and medical topics.

FIG. 26 shows an example histogram for evaluation protein expression.

FIG. 27 shows an example gating approach to evaluating protein expression.

FIG. 28 shows an example non-gating approach to evaluating protein expression.

FIG. 29 shows an example process flow for a non-gating approach to evaluating protein expression.

FIG. 30 shows another example process flow for a non-gating approach to evaluating protein expression.

FIG. 31 shows an example comparison between gating and non-gating approaches to evaluating protein expression.

FIG. 32 shows an example of a non-gating approach for evaluating expression of a complex phenotype.

FIG. 33 shows an example of a non-gating approach for protein expression applied to a Hodgkin's dataset.

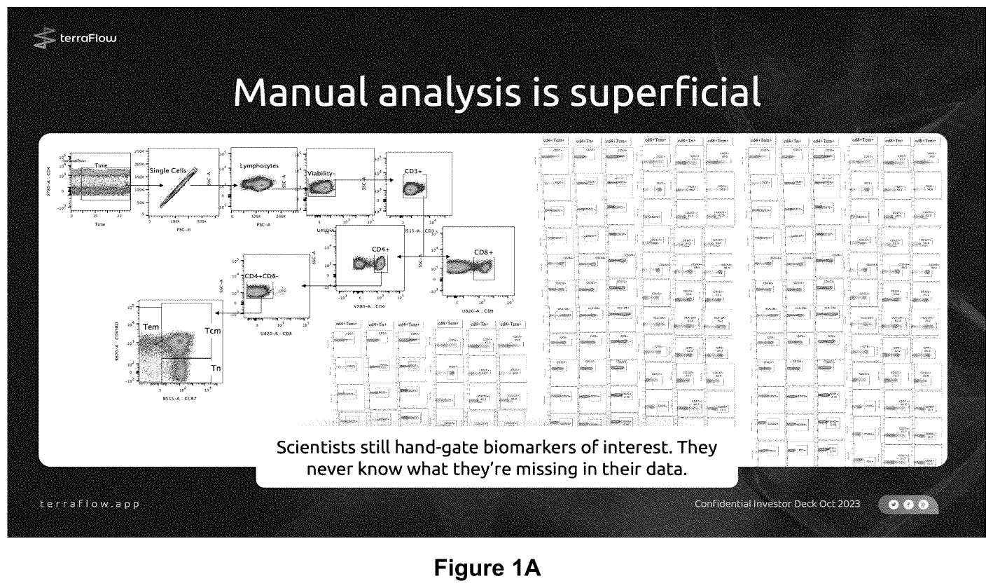

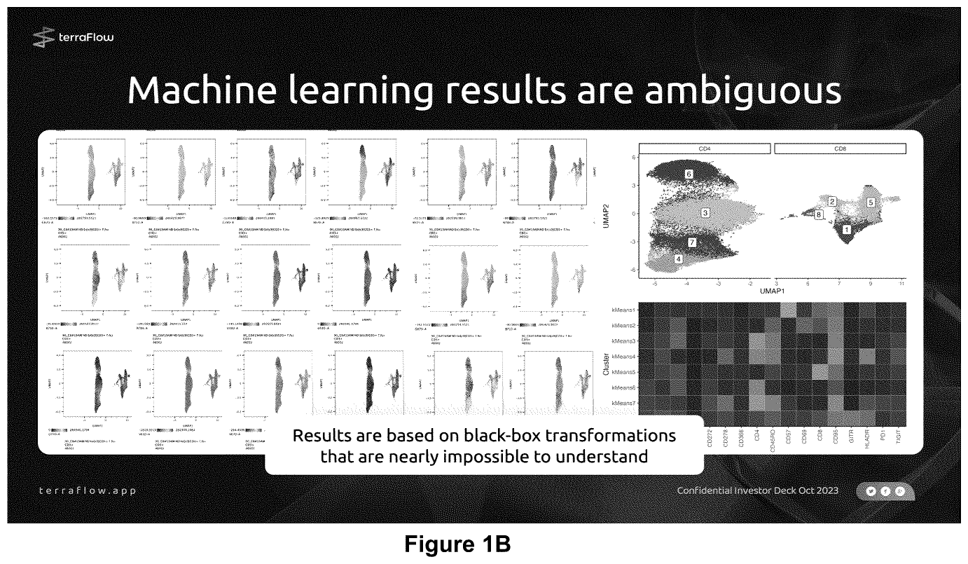

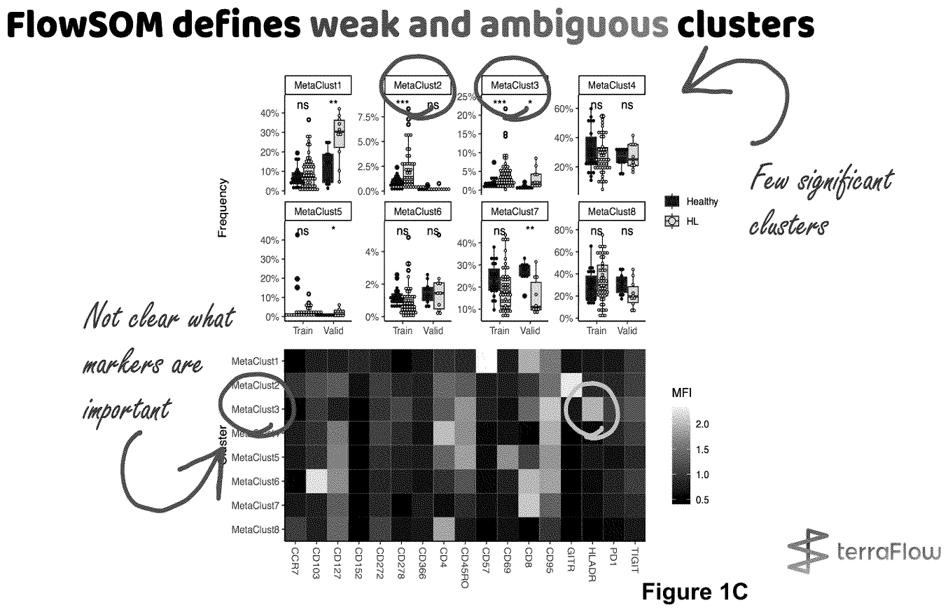

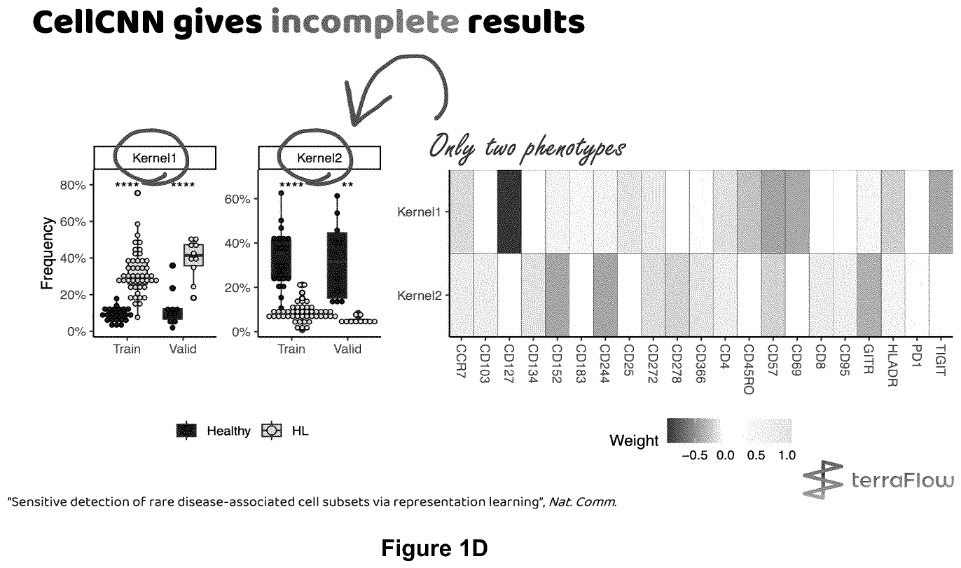

### DETAILED DESCRIPTION OF EXAMPLE EMBODIMENTS

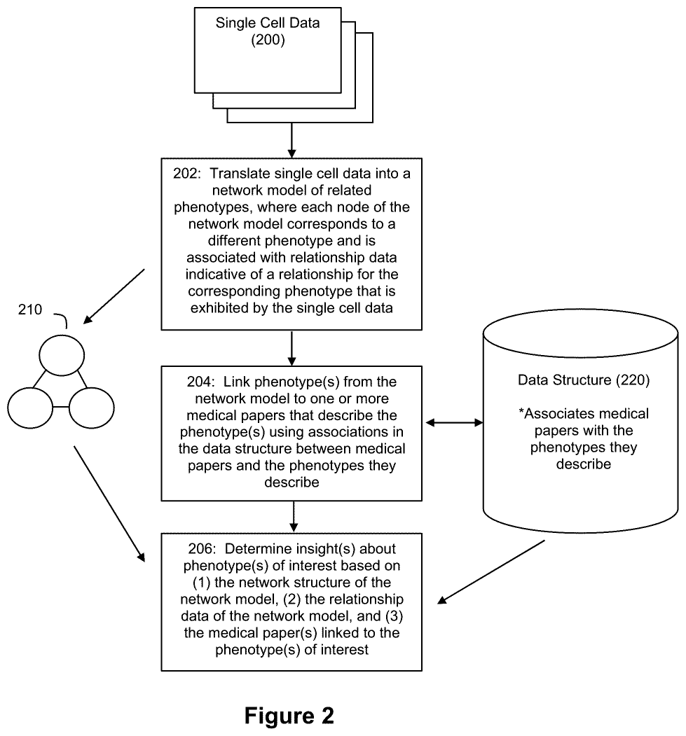

FIG. 2 shows an example process flow for transforming single cell data into one or more insights about the single cell data. The steps of FIG. 2 can be carried out by one or more processors, as discussed in greater detail below.

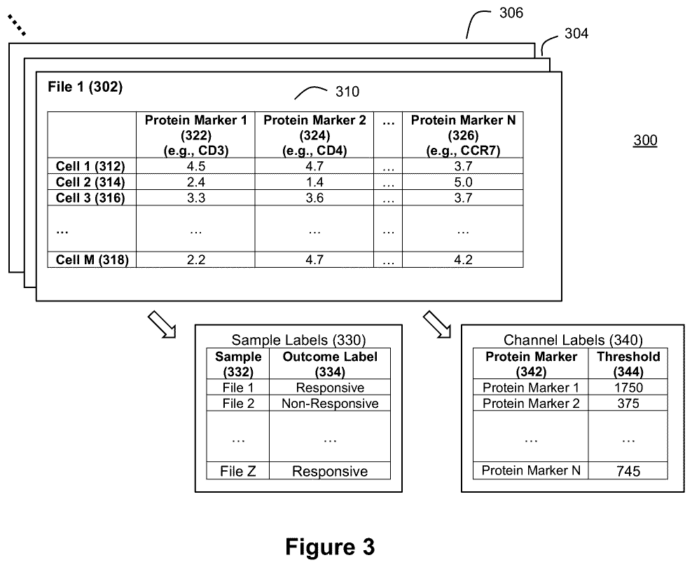

The process flow of FIG. 2 operates on single cell data 200. The single cell data 200 may take the form of flow cytometry data, shown by way of example in FIG. 3 as flow cytometry data 300. The flow cytometry data 300 can comprise a plurality of Flow Cytometry Standard (FCS) files 302, 304, 306, etc., which include single cell sample data 310. The single cell sample data 310 comprises measurements of whether a plurality of different protein markers (see 322, 324, 326, etc.) are present in different single cells of the samples (see 312, 314, 316, . . . , 318) that were analyzed by a flow cytometer. The samples can pertain to different subjects (e.g., human patients). The measurements can comprise measurements of fluorescent intensity for the protein markers in the cells. The single cell sample data 310 may take the form of a cell-by-protein matrix. A typical cell-by-protein matrix provides measurements for approximately 10,000 to 1 million cells with respect to approximately 12-40 protein markers However, it should be understood that more or fewer cells and/or protein markers may be encompassed by a cell-by-protein matrix in an FCS file. Moreover, it should be understood that each FCS file can be associated with a different patient and with an outcome for that patient (see 330).

Users can upload a cell-by-protein matrix as shown by FIG. 3 for each patient. They can also upload sample labels 330 (which can provide outcome labels 334, e.g., whether or not each patient (corresponding to the samples 332) responded to a treatment) and an optional threshold for each protein marker (see 340). As examples, the outcome labels can provide status data that indicates whether or not each patient (corresponding to the samples 332) was responsive or non-responsive to treatment, was diseased or not diseased (e.g., with respect to a disease classification), was treated or untreated by a treatment, etc. The thresholds 344 can be used to determine positive and negative expression of a given protein marker 342. However, as discussed below, it should be understood that other techniques could be used for determining positive and negative expressions of protein markers.

At step 202 of the FIG. 2 process flow, the single cell data 200 is translated into a data structure that defines a network model 210 of related phenotypes using the techniques described herein. Each phenotype is defined by a protein marker composition of one or more protein markers from the single cell data 200. For example, as discussed below, the phenotypes can be enumerated by forming all combinations of 1 to Q protein markers from among the protein markers in the single cell data. For example, given the following set of protein markers [CD4, CD8], the following phenotypes can be enumerated: [CD4+, CD4−, CD8+, CD8−, CD4−CD8−, CD4−CD8+, CD4+CD8−, CD4+CD8+]. In an example embodiment, the value of Q can be 5 so that all combinations of 1:5 protein markers are enumerated as phenotypes. However, it should be understood that other values for Q could be used by a practitioner if desired (e.g., Q=3, Q=4, Q=6, etc.). The maximum value of Q would be the number of protein markers in the dataset, which can be a larger number such as 40. The number of possible combinations increases with number of markers. However, fewer phenotypes are detectable as combinations become more complex. We find that Q<=5 is sufficient to capture most detectable phenotypes in a dataset. For a typical set of single cell data 200, this enumeration of phenotypes based on combinations of 1:5 protein markers can generate around 200,000 phenotypes.

The network model 210 comprises a plurality of nodes, where each node corresponds to a different phenotype among the enumerated phenotypes. Each node is also associated with relationship data that is indicative of a relationship for that node's corresponding phenotype with the defined outcome of the relevant patient sample. This relationship data can be derived from the protein marker-specific measurements in the single cell data 200. For example, the relationship data can take the form of a value indicative of a correlation between the node's corresponding phenotype with the defined outcome according to the single cell data 200. Further still, the nodes of the network model 210 are arranged in a network structure that is derived from relatedness between the protein marker compositions of the nodes' corresponding phenotypes as discussed above. The network structure can be defined through the connections that exist between the nodes of the network model 210. The network model 210 can be embodied as a data structure in memory of a computer system, such as a tree structure, graph structure, linked list, or a relational database structure that realizes the logical model defined by the network model 210.

FIG. 4 shows an example of a network model 210 produced as a result of step 202. Each node 402 in the network model 210 is associated with a corresponding phenotype and a relationship data value as noted above. Furthermore, connections 404 are added to the network model 210 to create edges that connect nodes 402 to each other according to the relatedness between the protein marker compositions of the node's corresponding phenotypes. For example, connections 404 can be added between nodes whose corresponding phenotypes differ by the addition of 1 protein marker or the removal of 1 protein marker. In this fashion, a node corresponding to the phenotype AB (comprising the combination of protein markers A and B) would be directly connected (a single degree of relatedness/connection) to nodes corresponding to the phenotypes A (the removal of protein marker B), B (the removal of protein marker A), ABC (the addition of protein marker C), ABD (the addition of protein marker D), etc. However, the node for phenotype AB would only be indirectly connected to the node for phenotype ABCD (through two degrees of relatedness/connection). Furthermore, the network model 210 can include a root node (not shown) that serves to represent the starting population containing all cells. The root node connects to nodes representing single-marker combinations, which connect to nodes representing two-marker combinations, and so forth. This ensures the network's completeness.

As discussed in greater detail below, the network structure of the network model 210 serves as a powerful contextual tool that allows the single cell data 200 to be meaningfully analyzed in a computationally efficient manner.

At step 204, one or more phenotypes from the network model 210 are linked to one or more medical papers that describe those one or more phenotypes. This linkage can be based on associations that exist in data structure 220. Data structure 220 associates a plurality of different medical papers with the phenotypes that are described in those medical papers. As an example, data structure 220 can take the form of an index that indexes a corpus of medical papers by the phenotypes that they describe. Accordingly, a given node 402 of network model 210 can be readily linked at step 204 to one or more medical papers by identifying the medical papers that are associated by the data structure 220 with the given node's corresponding phenotype.

At step 206, one or more insights are determined about one or more phenotypes of interest. These insight(s) are determined based on (1) the network structure of network model 210, (2) the relationship data exhibited by the network model for the phenotypes with respect to the relevant defined outcome, and (3) the medical paper(s) that are linked to the one or more phenotypes of interest. In some examples, the determined insight(s) may comprise a visualization that is derived from the network model 210, such as a visualization that depicts at least a portion of the network structure of the network model 210 along with indicators of noteworthy aspects of the single cell data 200 as reflected by one or more phenotypes of interest and information derived from the linked medical paper(s). In other examples, the determined insight(s) may comprise a natural language summarization of noteworthy aspects of the single cell data 200 as reflected by one or more phenotypes of interest and information derived from the linked medical paper(s).

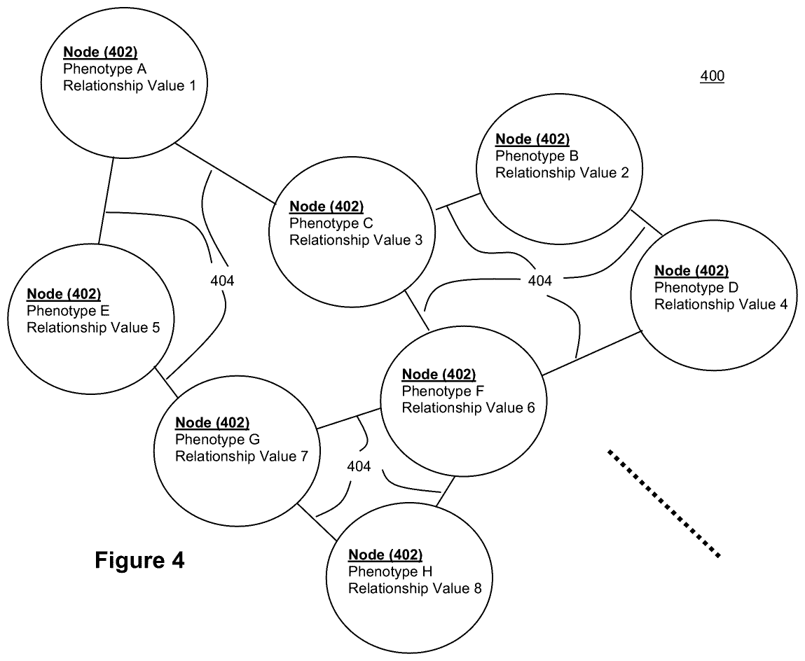

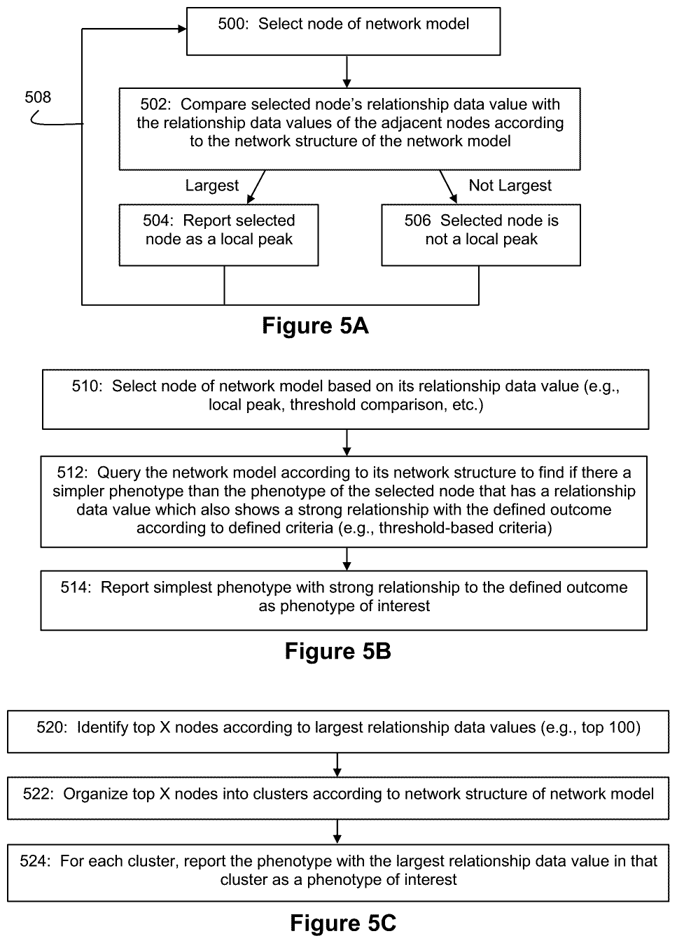

Any of a number of techniques can be used for identifying phenotypes of interest based on the network structure and the relationship data of the network model 210. FIGS. 5A-5C show examples of such techniques. The process flows of FIGS. 5A-5C can be performed as part of steps 204 and/or 206 of FIG. 2 if desired by a practitioner.

In the example of FIG. 5A, phenotype(s) of interest are identified by finding “local peaks” in the network model 210 whose correlation to the defined outcome is stronger than any of its adjacent nodes 402 in the network model 210. The extent of adjacency can be established by a defined depth of connections 404 that exist between a subject node and its adjacent nodes. For example, at a depth of 1, a subject node's adjacent nodes would include only those nodes that are directly connected to the subject node. As another example, at a depth of 2, a subject node's adjacent nodes would include only those nodes that are connected to the subject node by a maximum of 2 connections 404.

At step 500 of FIG. 5A, a node 402 from the network model 210 is selected. At step 502, the selected node's relationship data value is compared with the relationship data values of the adjacent nodes in the network model 210. As an example, the relationship data values can indicate an extent of correlation in the single cell data 200 as between the phenotypes and a defined outcome (e.g., whether the subject patient was responsive to a particular treatment). As noted above, the adjacency used at step 502 is a function of the network structure of the network model 210 so that a cluster of phenotypes evaluated at step 502 includes a set of phenotypes that are closely related to each other. By leveraging the network structure of the network model 210 in this fashion, the comparisons at step 502 are computationally-efficient. As a result of the comparisons at step 504, if the selected node exhibits the largest relationship data value, then the selected node can be reported as a local peak (step 504); and if the selected node does not exhibit the largest relationship data value, then the selected node is not a local peak (step 506). The process flow can then iterate back to step 500 where a next remaining node of the network model is selected. For example, the next selected node can be a node that was not a member of the node cluster that was considered at step 502. The phenotype(s) that correspond to the local peak(s) found as a result of the FIG. 5A process flow can serve as phenotypes of interest.

In the example of FIG. 5B, the network model 210 is searched to find the simplest phenotype(s) that exhibit a strong relationship with the defined outcome. It should be understood that simpler in this context as between two phenotypes is the phenotype that exhibits a fewer number of protein markers. Once again, the network structure of network model 210 allows the system to readily traverse the network model 210 to assess which nodes 402 correspond to simpler or more complex phenotypes by moving up or down the network structure of the network model 210.

At step 510 of FIG. 5B, a node 402 of the network model 210 is selected based on its relationship data. The selected node can be identified as a local peak (see FIG. 5A) or be identified using other criteria (such as a threshold comparison—e.g., select nodes whose correlation value is greater than 0.7). At step 512, the network model 210 is queried according to its network structure to find if there is a simpler phenotype than the phenotype corresponding to the selected node which has a relationship data value which also shows a strong relationship with the defined outcome. This determination can be made using defined criteria such as threshold-based criteria. For example, step 512 can identify a simpler phenotype as having a strong relationship with the defined outcome if the relationship data value of the simpler phenotype is within X % of the relationship data value of the subject phenotype, where X is chosen by a practitioner as a sufficiently high percentage to qualify as also exhibiting a strong relationship to the defined outcome (e.g., values of X in a range between 75% and 99% may be used, such as a threshold that requires the simpler phenotype to exhibit a relationship data value within 97% of the relationship data value for the subject longer phenotype). The threshold-based criteria may also require that the simpler phenotype satisfy a minimum threshold for relationship data value (e.g., a minimum correlation value of 0.7, etc.).

Simpler phenotypes relative to a subject node can be identified in the network model 210 by traversing the network model 210 through connections 404 to identify any nodes connected to the subject node 402 whose corresponding phenotypes exhibit a smaller number of protein markers. The simplest related phenotype that exhibits a strong relationship with the defined outcome can be reported as a phenotype of interest at step 514. For example, a node corresponding to phenotype CD95+CD4+ may exhibit a correlation value of 0.90; but the node corresponding to phenotype CD95+ may exhibit a correlation value of 0.88. Since CD95+ is a simpler phenotype than CD95+CD4+ and also exhibits a high correlation with the defined outcome, the process flow of FIG. 5B can operate to identify CD95+ as the phenotype of interest rather than CD95+CD4+. This traversal of the network model 210 according to its network structure to find relevant and simpler phenotypes can be advantageous because simpler phenotypes are easier to interpret and replicate in follow-on experiments. If a phenotype consists of just two markers, a user could replicate it using just two antibodies. Simpler phenotypes also tend to make more robust biomarkers because there are fewer biological variables involved. This is distinct from conventional ML workflows, which involve complex transformation of the entire feature set.

While the system can perform the process flows of FIGS. 5A and 5B separately if desired; it should also be understood that the process flow of FIG. 5B can be performed in conjunction with the process flow of FIG. 5A if desired. For example, the process flow of FIG. 5A can be performed to identify a set of phenotypes that serve as local peaks, and then the process flow of FIG. 5B can be performed on the local peaks to find out if there are simpler phenotypes that can better serve as phenotypes of interest.

FIG. 5C provides another example of how the network structure of the network model 210 can be leveraged to identify phenotype(s) of interest. In this example, targeted clusters according to the network structure can be mined to find phenotypes with strong relationships to the defined outcome.

At step 520 of FIG. 5C, the system identifies the top X nodes 402 of the network model 210 according to largest relationship data values. For example, step 520 can result in the system identifying the top 100 nodes according to largest relationship data values (although it should be understood that other values for X could be used, e.g., 50, 150, 200, etc.) At step 522, the system organizes the top X nodes into clusters according to the network structure of the network model 210. For example, step 522 can create the clusters based on which of the nodes 402 within the top X nodes are directly connected to each other via connections 404, where each distinct cluster is represented by interconnected nodes 402 among the top X nodes. At step 524, the system can find, for each cluster, the phenotype within that cluster with the largest relationship data value. Such phenotypes can be identified as phenotypes of interest. If desired, a practitioner can configure step 524 so that the system reports out the top Y phenotypes within each cluster as phenotypes of interest, where Y is a value greater than 1 (e.g., top 2, top 3, etc.).

Further still, it should be understood that a practitioner may choose to perform the FIG. 5C process flow in conjunction with the FIG. 5B process flow to identify phenotype(s) of interest, such as by running the FIG. 5C process flow to identify a set of phenotypes with large relationship data values, and then using the FIG. 5B process flow to find the simplest phenotypes that still exhibit strong relationships with the defined outcome.

As noted above and explained in greater detail below, these identified phenotypes of interest that are determined by the process flows of FIGS. 5A, 5B, and/or 5C can be used a basis for determining insights that are worthy of being presented to a user.

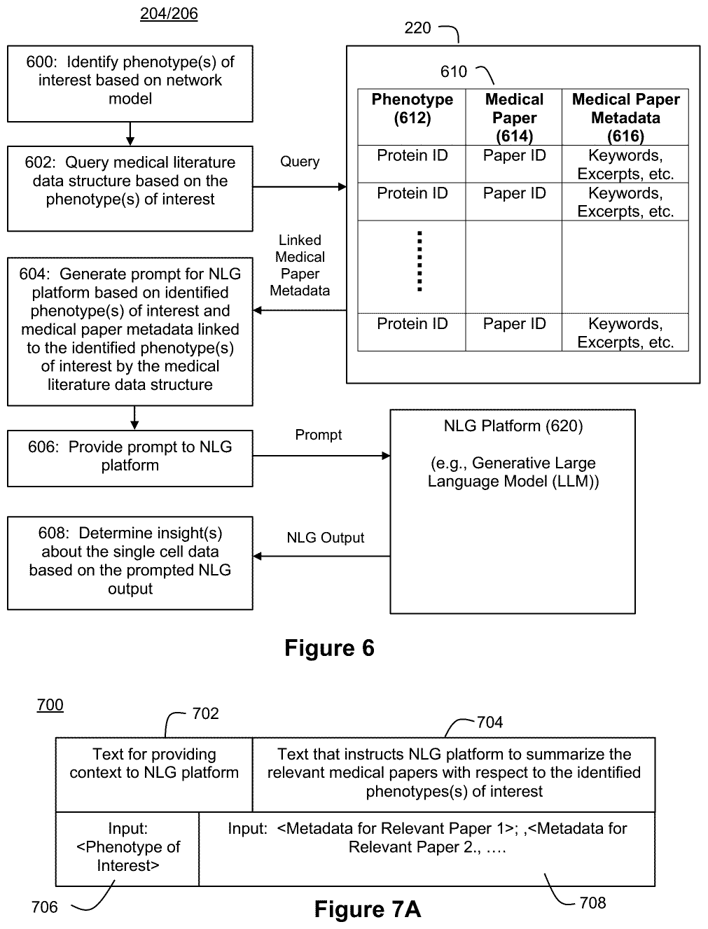

FIG. 6 depicts an example process flow that demonstrates how steps 204 and 206 of FIG. 2 can be carried out according to an example.

At step 600, the system identifies phenotype(s) of interest based on the network model 210. As noted above, the network structure of the network model 210 facilitates the analysis that results in determining which phenotypes qualify as phenotypes of interest.

Examples of techniques that can be used for carrying out step 600 are described in connection with FIGS. 5A-5C.

At step 602, the system queries the data structure 220 based on the identified phenotype(s) of interest. In this example, data structure 220 can comprise a medical literature index 610 that associates a plurality of different medical papers (see 614) with the phenotypes that are described in those papers (see 612). The phenotypes can be identified in the index 610 by a Protein ID (such as a standardized protein identifier (e.g., a UniProt ID)). The medical papers can be identified in the index 610 by a paper identifier. As an example, the Paper ID can take the form of a digital object identifier (DOI) or the like for a subject medical paper. Further still, the data structure 220 can associate the phenotype-relevant medical papers with metadata about the phenotype-relevant medical papers (see 616). The metadata 616 may comprise keywords for the subject medical papers, titles for the subject medical papers, excerpts from the medical papers (e.g., passages from the medical papers that pertain to the subject phenotype; abstracts of the medical papers; etc.), scores that indicate how strongly the papers relate to the subject phenotype, and/or other aspects of subject medical papers. In some embodiments, the metadata 616 may include the full text of the medical papers if desired by a practitioner. It should also be understood that a given phenotype (Protein ID) may be associated with multiple different medical papers, in which case index 610 can include multiple entries for the same Protein ID, where each entry corresponds to a different linked medical paper. Additional details about how the data structure 220 can be created are discussed in greater detail below.

Thus, step 602 can involve querying the data structure 220 using an identified phenotype of interest to retrieve medical paper metadata 616 that is linked by the data structure 220 to the queried phenotype of interest. The response from the data structure 220 can also include the Paper ID(s) of the medical paper(s) that are linked by the data structure 220 to the queried phenotype of interest.

At step 604, the system generates a prompt for a natural language generation (NLG) platform 620 based on the identified phenotype(s) of interest from step 600 and the linked medical paper metadata obtained as a result of step 602. The NLG platform 620 can comprise a computer system or executable software code that is configured to generate a natural language output in response to an input prompt. As an example, the NLG platform 620 may comprise a generative artificial intelligence (AI) system, such as a generative large language model (LLM). The generative LLM can be trained based on large corpuses of medical literature, particularly medical literature in the relevant fields (e.g., flow cytometry, immunophenotyping, immunology, etc.) so that it develops a robust knowledge base in the relevant fields. For example, OpenAI's GPT-40 can be used as the NLG platform 620 if desired by a practitioner. Examples of other NLG platforms 620 that a practitioner may choose to leverage can include other LLMs such as Claude and Mistral, as well as open-source models such as LLaMa and BERT and their domain-specific derivatives such as BioLLaMa and BioBERT, or even custom-designed NLP and NLG applications. The models can be fine-tuned on literature that has been manually curated by an expert reviewer. The prompt that is generated at step 604 can be configured to request that the NLG platform 620 summarize the linked medical papers as they relate to the identified phenotype(s) of interest.

To support the generation of a suitable prompt, step 604 can leverage a prompt template that comprises natural language text in combination with a plurality of variable placeholders that get populated with specific data on each instance of step 604 being performed. By using prompt templates in this fashion, the prompt templates can be re-used and instantiated each time that system is operated to determine insights with respect to a set of single cell data without requiring re-coding or manual intervention by system users to adapt the system for the new set of single cell data.

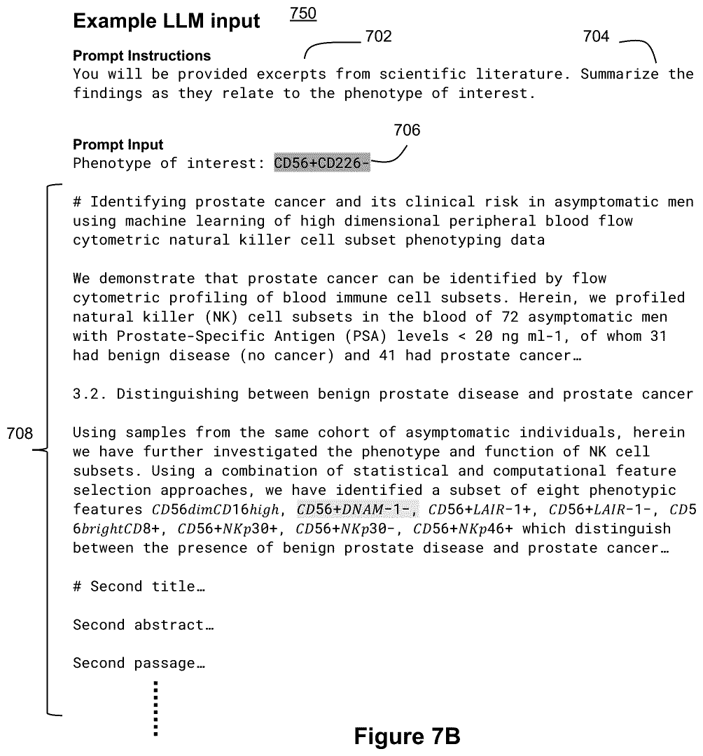

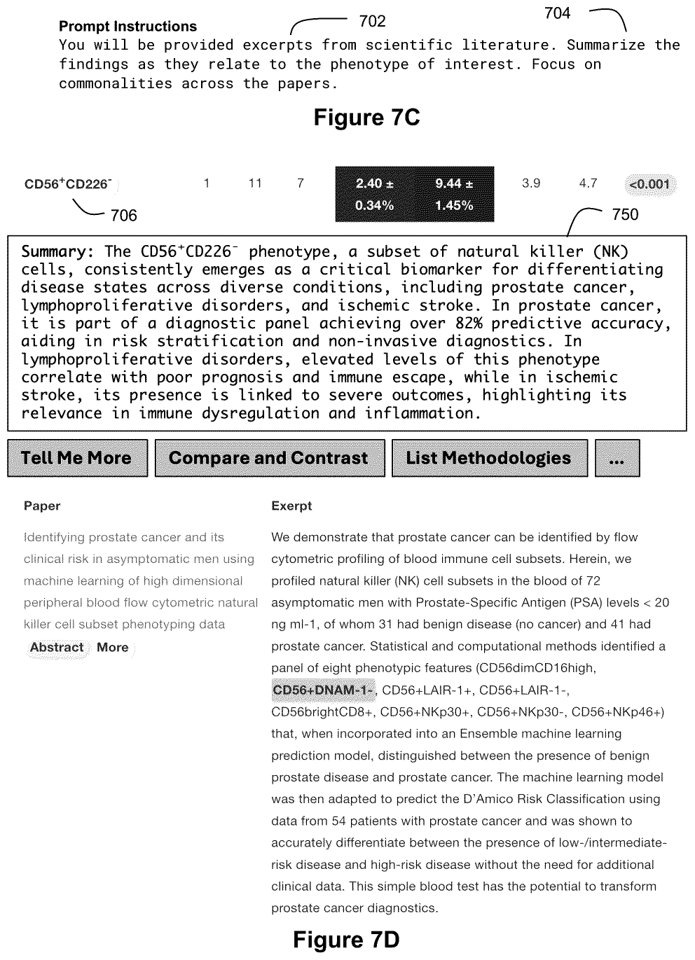

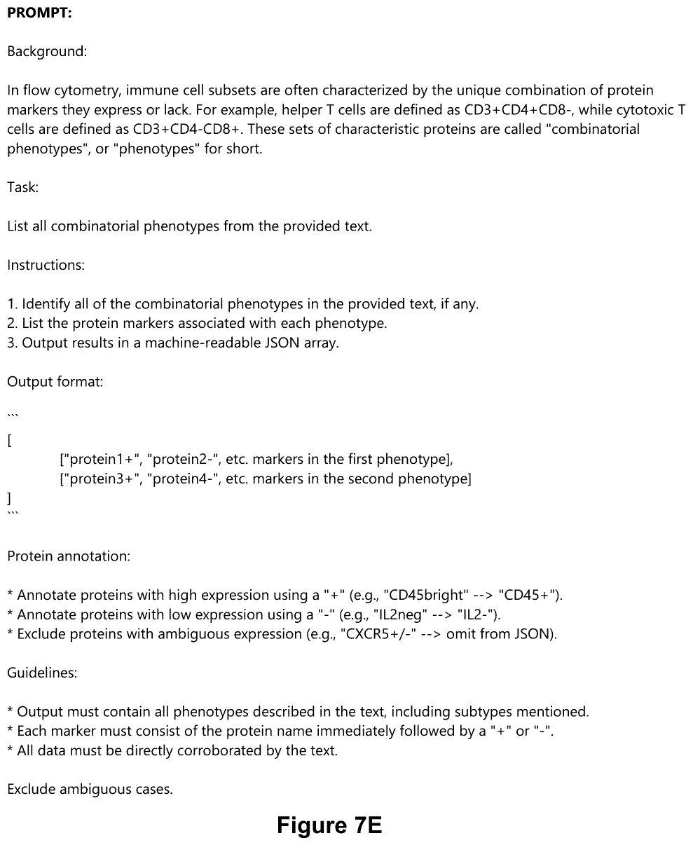

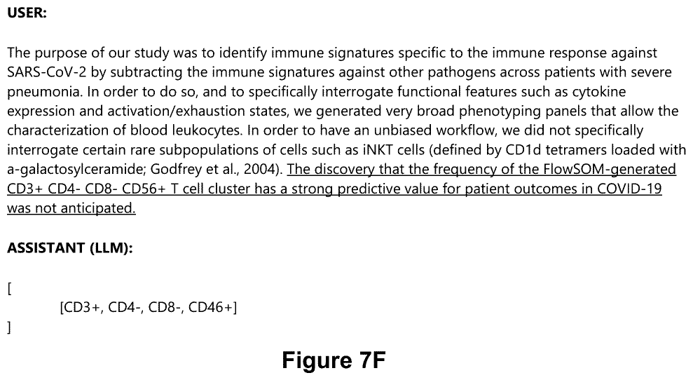

FIG. 7A depicts an example prompt template 700 that can be used in the operation of step 604. The prompt template 700 includes a text section 702 that provides the NLG platform 620 with context for the requested output and a text section 704 that provides the NLG platform 620 with command(s) regarding the nature of analysis it wants to have reflected in the requested output. The prompt template 700 also includes placeholders 706 and 708. Placeholder 706 is a field through which the relevant phenotype of interest is specified, and placeholder 708 is a field (or fields) through which the relevant medical paper metadata is provided to the NLG platform 620. It should be understood that placeholder 708 may include metadata for a plurality of different medical papers that were found at step 602 to be relevant to the identified phenotype of interest. FIG. 7B depicts an example prompt 750 that can be derived from the prompt template 700, where the identified phenotype of interest is “CD56+CD226−” and where the prompt 750 requests that the NLG platform 620 operate to “Summarize the findings [of the input scientific literature] as they relate to the phenotype of interest”. It should also be understood that text section 704 need not be limited to one type of analysis. For example, as shown by the example of FIG. 7C, the text section 704 can specify multiple types of analysis that it wants to be reflected in the requested output. With respect to FIG. 7C, it can be seen that text section 704 not only requests a summarization of the specified literature, but it also requests that the NLG platform “Focus on commonalities across the papers”. As such, it should be understood that the prompt 700 and NLG platform 620 can support operational commands such as “Summarize”, “Focus”, “Explain”, “Compare and Contrast”, “Highlight Discrepancies”, “List Methodologies”, etc., or compound combinations thereof.

At step 606, the system provides the generated prompt to the NLG platform 620. The system can include an application programming interface (API) that interfaces the computer system that performs steps 604 and 606 with the NLG platform 620. The API can define a structured request for interfacing with an external server (e.g., an OpenAI server on which the NLG platform 620 runs) or an internal server acting as the NLG platform 620 and running an LLM. The structured request can comprise a “chat” that includes a system prompt and a literature excerpt. The NLG platform 620 can extract phenotype data from the excerpt and extend the chat via the API with a machine-readable JSON array. FIGS. 7E and 7F show an example specification for an API through which prompts can be provided to an NLG platform 620. Through such an API, in response to the received prompt, the NLG platform 620 produces an NLG output that satisfies the prompt. As noted above, the NLG platform 620 is previously trained on large corpuses of relevant medical literature so that it is capable of performing requested operations that involve interpreting, analyzing, summarizing, etc. the phenotype-relevant medical papers.

At step 608, the system determines one or more insights about the phenotype(s) of interest and the single cell data 200 based on the prompted NLG output. For example, the system can use the NLG output as the determined insight(s), and this NLG output can be presented to a user via a user interface (e.g. presented on a display screen, etc.). FIG. 7D shows example GUI that can present a sample NLG output 750 that can be produced by the NLG platform 620 and presented to a user as a result of step 608 in response to the prompt shown by FIGS. 7B and 7C. The sample NLG output 750 provides a natural language summarization of the phenotype-of-interest 706 with respect to phenotype-relevant medical literature as discussed herein. The example GUI shown by FIG. 7D can also include user-selectable buttons for the user to access additional NLG output, such as a “Tell Me More” button and a “Compare and Contrast” button, which can operate to either trigger new prompts to the NLG platform 620 that are geared toward these requests or present previously-generated NLG outputs relating to these requests. The GUI that presents the output to the user can also provide statistics/data points about the phenotype of interest as well as snapshots of the relevant medical paper(s) from which the summarization 750 was generated (e.g., title and excerpt information for the relevant paper(s)).

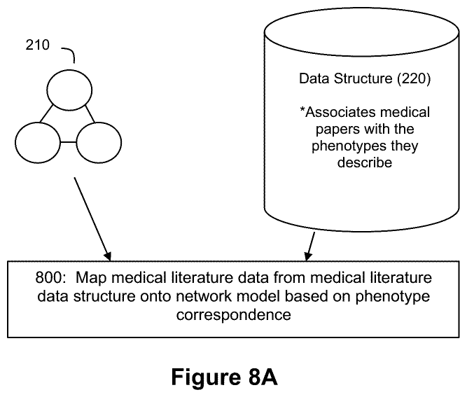

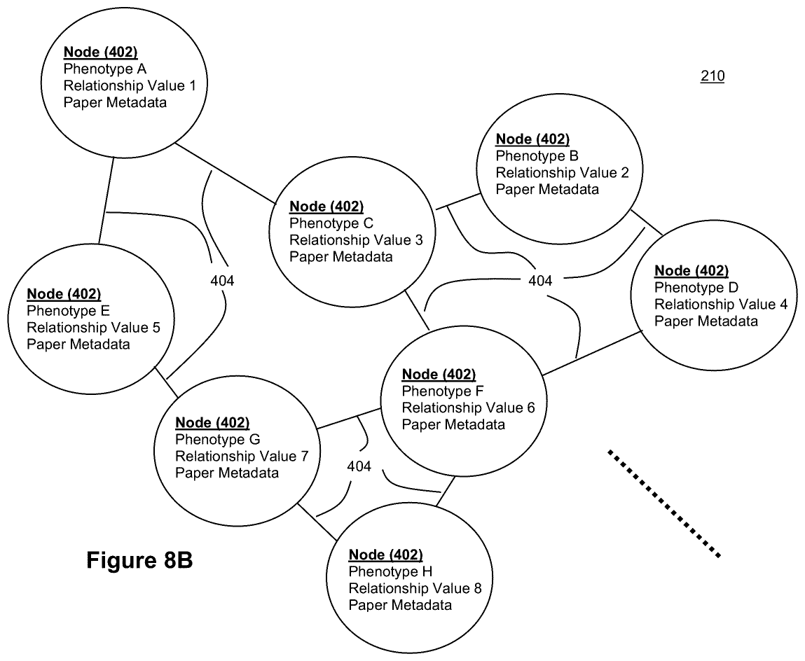

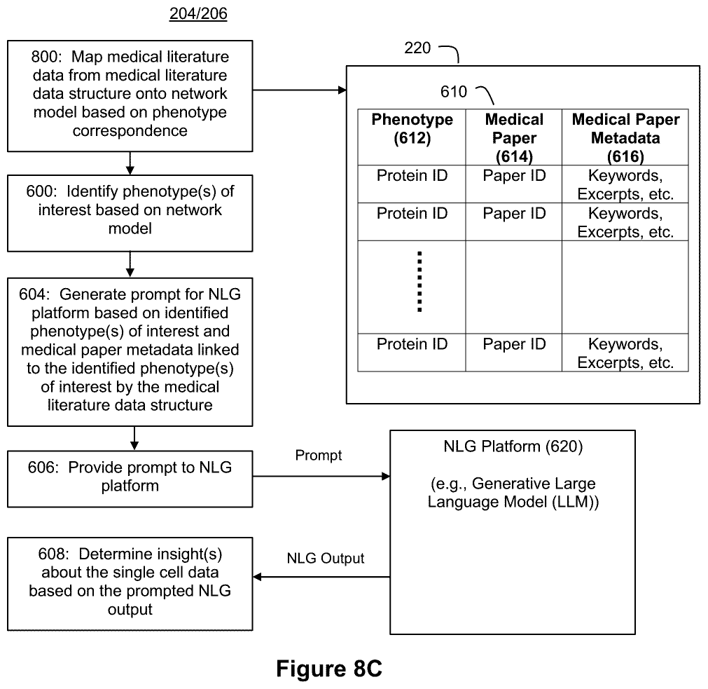

In some embodiments, a practitioner may find it desirable to perform step 202 of FIG. 2 on an “as needed” basis when a new analysis of single cell data is desired (such as performing step 202 for phenotypes of interest that are identified using techniques such as those shown by FIGS. 5A-5C, while leaving the other phenotypes of the network model 210 unlinked to relevant medical literature). However, in other embodiments, a practitioner may find it desirable to link all of the phenotypes (or at least more than just the phenotypes that have been identified as phenotypes of interest) to relevant medical literature. For example, this approach may be desirable in embodiments where a practitioner wants to use linkages to relevant medical literature among the criteria that are used for identifying which phenotypes qualify as phenotypes of interest. FIG. 8A shows an example process flow where step 202 can include a mapping operation 800 that maps medical literature data from the data structure 220 onto the network model 210. This mapping can use correspondence between phenotypes (phenotypes in common) between the network model 210 and the data structure 220 as the linking key. For example, a given node 402 of network model 210 that corresponds to Phenotype X can be augmented with additional data derived from the medical paper(s) linked by the data structure 220 to Phenotype X. Step 800 can be performed for a plurality of the phenotypes which have corresponding nodes 402 in the network model 210 (such as performing the mapping for all of the nodes 402 of the network model 210).

FIG. 8B shows an example network model 210 whose nodes 402 have been augmented as a result of step 800 by also associating the nodes 402 with medical paper metadata from the medical paper(s) linked by the data structure 220 with each node's corresponding phenotype. This metadata can include metadata such as the types of metadata that were described for index 610 in FIG. 6 (see 616). The medical paper metadata added to the nodes 402 can also include aggregated medical paper metadata, such as counts of how many different medical papers are linked to a subject phenotype and one or more scores that quantify one or more characteristics of the linked medical papers (e.g., a notability score, relevance score, specificity score, and/or aggregations thereof as discussed below), etc.

FIG. 8C shows another example process flow for steps 204 and 206 of FIG. 2 where a mapping operation 800 is employed. With this example, the mapping step 800 can be performed to augment the network model 210 with information derived from the phenotype-relevant medical papers (as linked via data structure 220). Step 600 can then operate to identify phenotype(s) of interest based on an analysis of the network model 210 which has been augmented with information from the phenotype-relevant medical papers. For example, phenotypes that show a moderate to strong relationship with the defined outcome and are strongly reported in medical literature as indicated by the augmented network model 210 can qualify as phenotypes of interest that get reported to the user (if desired by a practitioner). Similarly, phenotypes that are weakly reported in the medical literature but show a moderate to strong relationship with the defined outcome can also qualify as phenotypes of interest that get reported to the user (if desired by a practitioner). From step 600, the process flow can perform steps 604, 606, and 608 as discussed above in connection with FIG. 6.

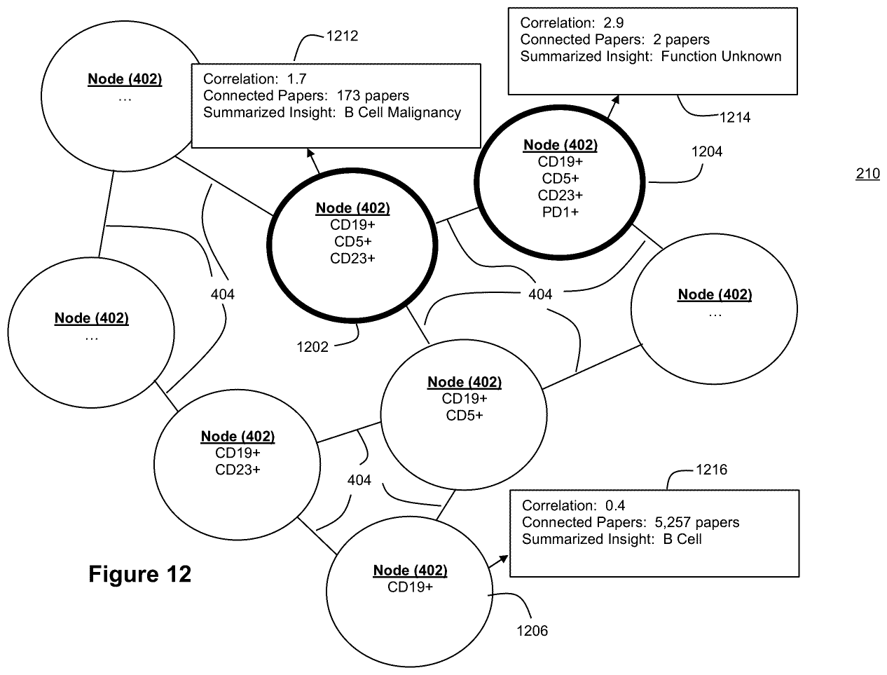

Moreover, the system can also map the insights that are determined at step 608 onto the network model 210 if desired. In this fashion, the nodes of the network model 210 can be further augmented with the insight(s) that are determined as a result of step 608. Thus, if Insight A is determined at step 608 for Phenotype X (“Phenotype X is known to contribute to an immune response to Y”), then the node 402 for Phenotype X in the network model can be augmented with Insight A so that Insight A becomes associated with the node 402 corresponding to Phenotype X (e.g., FIG. 12).

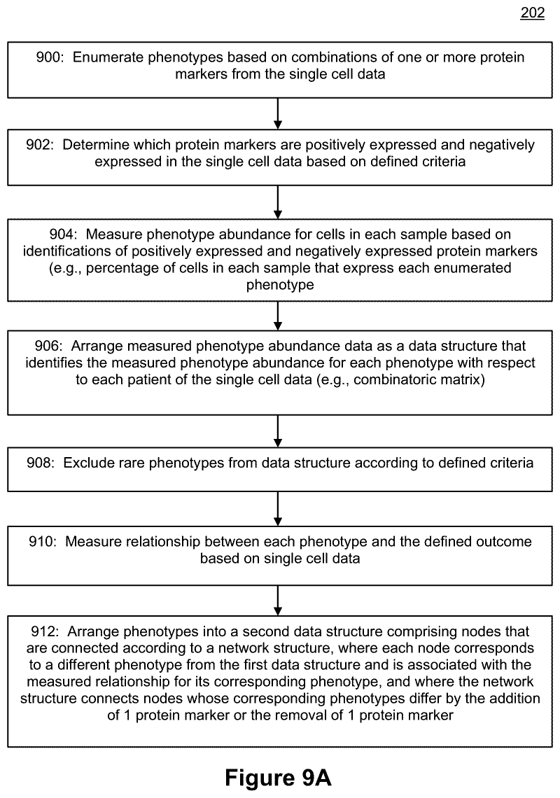

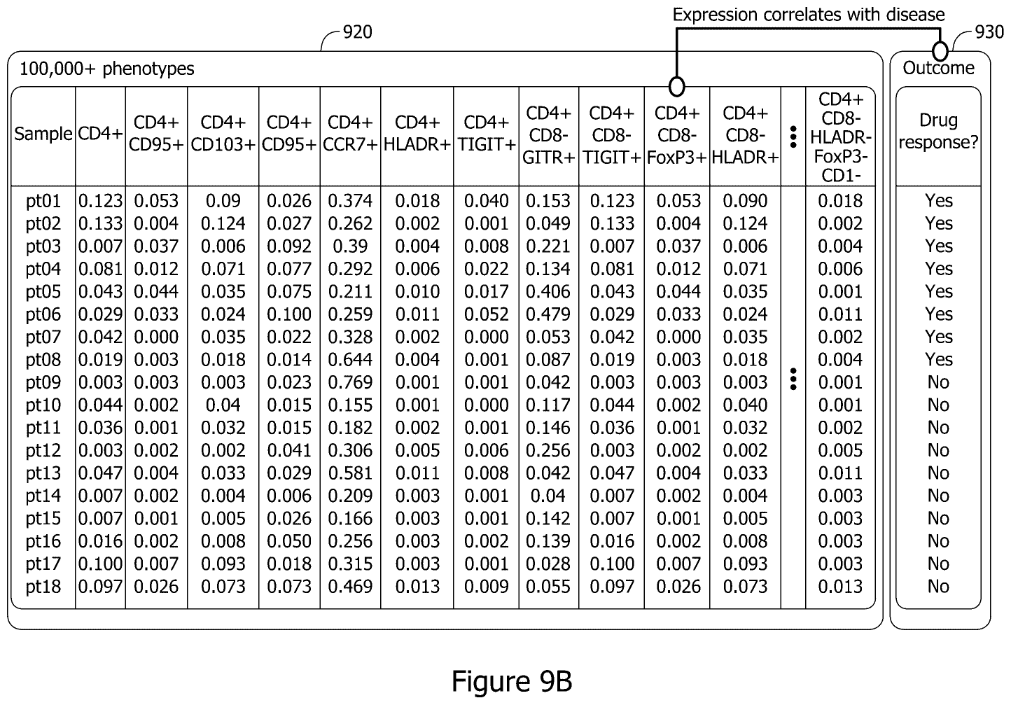

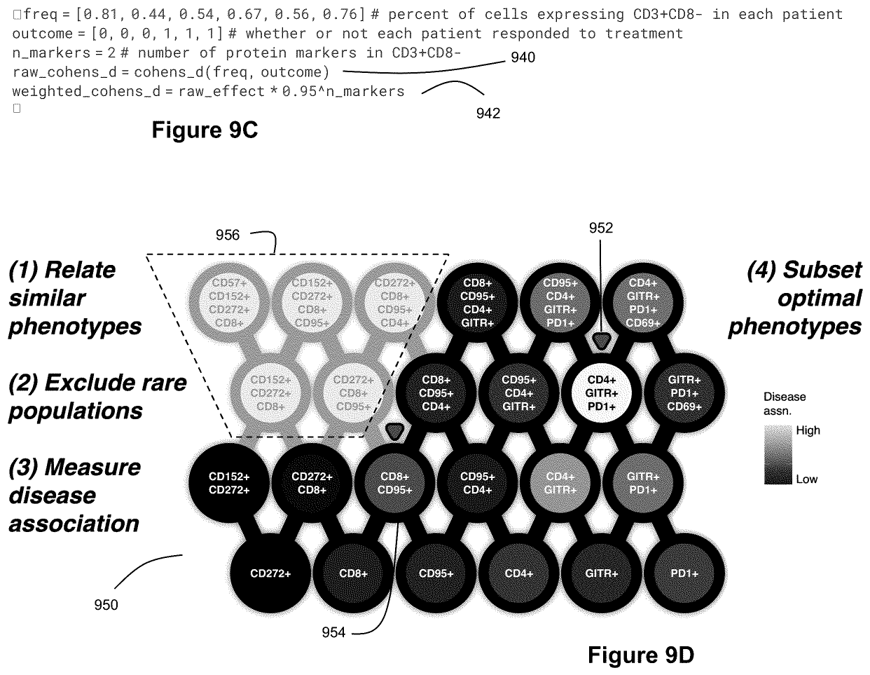

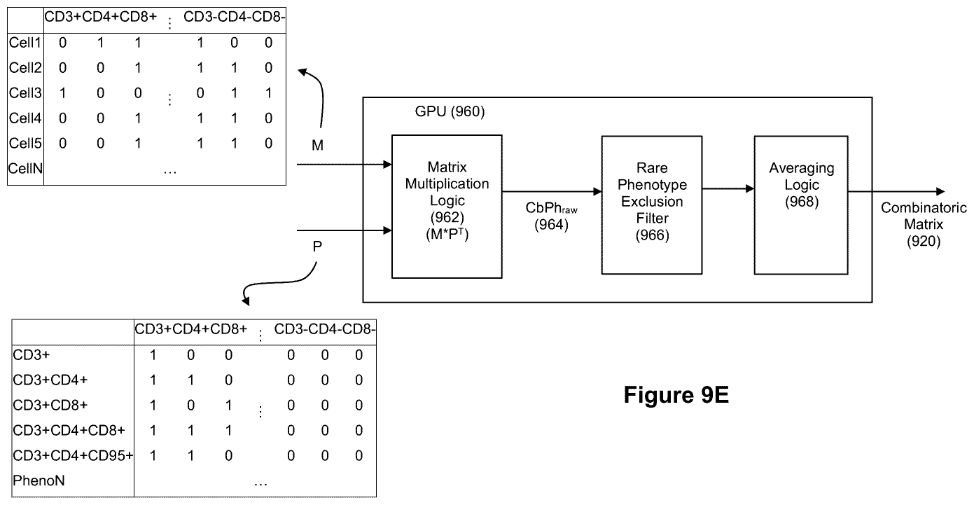

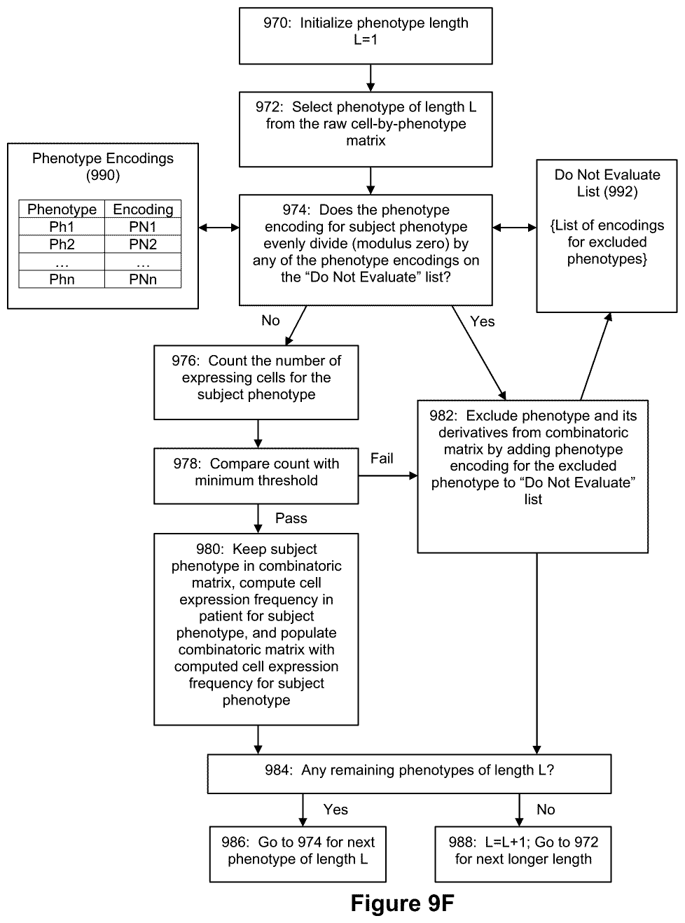

FIG. 9A shows an example process flow for translating single cell data 200 into the network model 210 according to step 202 of FIG. 2. At step 900, the system identifies the different protein markers that are covered by the single cell data 200 and enumerates the phenotypes based on these protein markers. As noted above, step 900 can form all combinations of 1 to Q protein markers from among the protein markers in the single cell data 200 (where Q can be a value such as 5, as noted above). For a typical set of single cell data 200, this enumeration of phenotypes based on combinations of 1:5 protein markers can generate around 200,000 phenotypes.

At step 902, the system determines which protein markers are positively expressed and negatively expressed within each sample of the single cell data 200 based on defined criteria. These criteria can include gating criteria or non-gating criteria depending on the desires of the practitioner. For example, as gating criteria, step 902 can employ so-called manual gates where the user provides thresholds that define positive and negative expressions for each protein marker (e.g., see FIG. 27). For example, these thresholds can be fluorescent intensity cutoffs for each protein marker that govern whether a given protein marker qualifies as positively expressed or negatively expressed in a sample. Channel labels 340 as shown by FIG. 3 can be provided by a user to define such gating criteria. As another example of gating criteria, step 902 can employ system-defined gating thresholds that are automatically imputed to the protein markers. To support such system-defined gating, the thresholds can be imputed using the openCyto library ((e.g., see Jiang, Mike, “OpenCyto: How to use different auto gating functions” available online at https://bioconductor.org/packages/devel/bioc/vignettes/openCyto/inst/doc/HowToAutoGating.html). A practitioner may also choose to use non-gating criteria to determine which protein markers are positively and negatively expressed in the single cell data samples. Additional details regarding example non-gating approaches are discussed in greater detail below.

At step 904, the system performs a combinatoric evaluation of the single cell data 200 with respect to the phenotypes that are deemed present in the cells according to which of the protein markers are deemed as positively expressed and negatively expressed in the cells. To perform this combinatoric evaluation, the system can measure the abundance of phenotypes for cells in each sample based on the identifications of positively expressed and negatively expressed protein markers. For example, the measured abundance can be a percentage of cells in each sample that express each enumerated phenotype. A phenotype can be deemed expressed by a cell if all of its component protein markers are present in the cell according to positive expression or negative expression. For example, the phenotype CD4+CD8− would be deemed present in a cell if that cell included a positive expression of CD4 (CD4+) and a negative expression of CD8 (CD8−). At step 906, the measured phenotype abundance data can be arranged as a data structure that identifies the measured phenotype abundance for each phenotype with respect to each patient of the single cell data 200. This data structure can take the form of a combinatoric matrix 920 such as that shown by FIG. 9B. The matrix 920 of FIG. 9B includes a first dimension that corresponds to the different patients of the single cell data (e.g., see the rows for the matrix 920, which correspond to different patient samples) and a second dimension that corresponds to the different phenotypes (e.g., see the phenotype columns for the matrix 920). Each cell of the matrix 920 can be populated with the measured phenotype abundance for each patient-phenotype combination. As noted above, the measured phenotype abundance data can be percentage values for the percentage of cells in each patient sample in which the subject phenotype was detected. FIG. 9B further shows how each sample can be linked to an outcome label 930 (e.g., a label that indicates whether each patient was responsive to a particular drug treatment).

This combinatoric evaluation can be computationally-demanding. However, to provide technical improvements in computing performance (e.g., reduced latency/higher speed), the combinatoric evaluation can employ matrix operations and parallel processing via parallelized compute resources such as graphics processing units (GPUs) as explained in greater detail below.

At step 908 of FIG. 9A, the system can exclude rare phenotypes from the data structure 920. The determination of which phenotypes qualify as rare can be based on defined criteria. The number of possible phenotypes increases factorially with the number of protein markers in the single cell data 200. However, fewer phenotypes are detectable in the single cell data 200 as the number of protein markers that make up the phenotype increases (more complex phenotypes tend to be rarer). The process of building the network model 210 can exclude phenotypes that are represented by fewer than a defined minimum threshold in the single cell data 200. For example, the defined minimum threshold can exclude phenotypes that are detected in fewer than X cells per sample on average. The value of X can be chosen by a practitioner according to the empirical needs and desires of the practitioner, but as an example, a value of 25-75 cells per sample on average can be used as the minimum threshold (e.g., X=50). Using X=50 as an example, this would mean that a phenotype can be represented by 100 cells per sample in one group and totally absent in another group, so long as the average is at least 50 cells per sample across the subject single cell samples that make up the single cell data 200. Furthermore, it should be understood that adding protein markers to make phenotypes longer will always operate to decrease phenotype abundance, while removing protein markers to make phenotypes shorter will always operate to either increase phenotype abundance (or at least leave phenotype abundance unchanged).

At step 910, the system measures a relationship between each remaining (non-excluded) phenotype and the defined outcome in view of the single cell data 200. The measured relationship for each phenotype can be represented by a relationship data value for each phenotype. This measured relationship can quantify a relationship such as how well each phenotype correlates with a defined outcome for the patients of the single cell samples within the single cell data 200. Step 910 can use the measured abundance for each phenotype in each patient (see matrix 920 in FIG. 9B) to calculate a correlation measure as between each phenotype and patient outcome. As an example, Cohen's d can used as the measure of correlation (see 940 in FIG. 9C), although other measures of effect size such as Pearson's correlation or log 2 fold change can be used here as well.

Further still, the measured relationship can further weight the relationship data value based on the number of protein markers that make up the phenotype. For example, step 910 can penalize the relationship or effect size based on the number of protein markers that are included in the phenotype so that longer phenotypes (more protein markers) are penalized as compared to shorter phenotypes (fewer protein markers). For example, the weighted relationship data value can be computed as the raw relationship value multiplied by a scalar that is weighted as a function of how many markers are present in the phenotype (e.g., see 942 in FIG. 9C). Simpler phenotypes are not only easier to interpret but are also more likely to validate to new data (fewer measurements=less noise). As an example, consider a three-marker phenotype such as CD4+GITR+PD1+. Using the penalty factor of FIG. 9C, the correlation value for this phenotype would be scaled by a factor of 0.95*3 (=0.86). While the example of FIG. 9C employs a 95% scalar, it should be understood that other scalar values could be employed if desired by a practitioner (e.g., 0.85, 0.90, 0.925, 0.975, etc.).

At step 912, the system arranges the phenotypes and their computed relationship data values into a data structure that serves as the network model 210. As noted above, the network model 210 comprises a plurality of nodes 402, and each node 402 corresponds to a different phenotype among the phenotypes. Moreover, as noted above, the nodes 402 are arranged in the network model 210 according to a network structure that is based on the relatedness between the nodes' corresponding phenotypes. In an example embodiment, the network structure is defined so that connections 404 are created between nodes 402 whose corresponding phenotypes differ by the addition or removal of a single protein marker. With such a network structure, the network model 210 exhibits a tree-like structure where a first level of the network model 210 contains nodes corresponding to phenotypes that exhibit a single protein marker, a second level of the network model 210 contains nodes corresponding to phenotypes that exhibit two protein markers (with connections 404 being made between the nodes 402 whose phenotypes differ by 1 protein marker), and so on for a third level, fourth level, etc. As an example, with this approach, the network model 210 would have connections 404 (edges) that connect nodes 402 corresponding to the following chain of phenotypes—CD4+→CD4+CD95+→CD4+CD95+GITR+. Further still, the nodes 402 of the network model 210 can be associated with the relationship data values computed at step 910 for their corresponding phenotypes.

FIG. 9D shows an example network model 950 that can be created as a result of step 912. Moreover, it should be understood that some practitioners may find it desirable for the system to produce a visualization of the network model 210 created as a result of step 912 for presentation to a user. With such a visualization, the nodes can be color-coded or otherwise highlighted based on the relationship data values of their corresponding phenotypes. In this example, the measured relationship is correlation with the defined outcome. It can be seen from FIG. 9D that node 952 (for CD4+GITR+PD1+) exhibits a strong correlation with the defined outcome and that node 954 (for CD8+CD95+) exhibits a moderate correlation with the defined outcome. Furthermore, FIG. 9D shows a set of nodes/phenotypes that were excluded as rare phenotypes as a result of step 908 (see 956 in FIG. 9D).

Because a visualization such as that shown by FIG. 9D shows the network structure of the network model 950, the visualization captures gradient changes in effect size as protein markers are successively added, removed, and/or swapped between related phenotypes. This powerful manner of communicating the single cell data 200 provides users with context for understanding which phenotypes are interesting or noteworthy. For example, while node 954 has a moderate effect size, its position in the network structure as a “local peak” which can be visually verified by a user to allow a user to quickly identify the phenotype corresponding to node 954 as a phenotype of interest (e.g., where the phenotypes corresponding to nodes 952 and 954 can be characterized as two local peaks that may represent unique differences between patient groups. Further still, the visualization of the network model 210 can include displays of the phenotype-related medical paper information (which may include summarizations of the phenotype-related medical paper information) as shown by FIGS. 8B and 12. As such, it should be understood that a visualization of the network model 210 may itself serve as an insight that is determined by the system at step 206. However, other practitioners may find it more desirable to alternatively or additionally generate a natural language output as demonstrated by the FIG. 6 process flow.

While the FIG. 9A process flow includes step 908 to exclude rare phenotypes from the network model 210, it should be understood that some practitioners may choose to omit step 908. For example, in scenarios where a practitioner perceives little benefit from excluding rare phenotypes (e.g., where the computational load of including the rare phenotypes in the network model 210 is expected to be minimal; where a practitioner may be interested in evaluating rare phenotypes, etc.), the network model 210 can be created from the single cell data 200 without performing step 908. Moreover, while the example of FIG. 9A shows that step 910 is performed before step 912, it should be understood that a practitioner may choose to implement the process flow in a manner where step 912 is performed before step 910.

As noted above, the FIG. 9A process flow can be accelerated by offloading one or more of the processing steps of the FIG. 9A process flow to highly parallelized compute resources such as GPUs. Thus it should be understood that one or more parallelized compute resources such as one or more GPUs can serve as an offload engine for one or more other processors that may be used by the system to process the single cell data 200.

For example, steps 904 and 906 of FIG. 9A can be offloaded to a GPU 960 as shown by FIG. 9E to leverage the GPU's powerful parallelized architecture for fast arithmetic operations. As shown by FIG. 9E, the GPU can be fed with a cell-by-marker matrix (M) and a phenotype-by-marker matrix (P). These matrices M and P can serve a model of the single cell data 200 that will get transformed by the GPU 960 into a new model of the single cell data 200 as discussed below.

The cell-by-marker matrix M can be constructed as a result of step 902, where each cell of the matrix can indicate whether a given protein marker was found to be expressed in that cell. In this context, positive and negative expressions for protein markers can be considered different protein markers (e.g., CD4+ and CD-would be considered different markers in matrix M). The values in the matrix M can be Boolean in nature (e.g., where “1” indicates cell expression by the subject protein marker and where “0” indicates lack of cell expression by the subject protein marker). However, as discussed in greater detail below, the values in matrix M need not be Boolean. For example, in a non-gating approach to evaluating protein expression as discussed below, the values in matrix M can be continuous sigmoid-transformed intensity values.

The phenotype-by-marker matrix P can be constructed where the rows correspond to the enumerated phenotypes (see step 900) and where the rows correspond to the different individual protein markers that are present in the single cell data 200. The values in each cell of the matrix can indicate whether each protein marker is present or absent in the corresponding phenotype (e.g., present=“1”; absent=“0”).

The GPU 960 can include matrix combinatorial logic and aggregation logic that operates on input matrices M and P and data derived therefrom. For example, the matrix combinatorial logic can take the form of matrix multiplication logic 962; and the aggregation logic can take the form of a rare phenotype exclusion filter 966 and averaging logic 968 as shown by FIG. 9E. However, it should be understood that if a practitioner does not want to exclude rare phenotypes from the analysis, the GPU 960 can omit the filter 966 if desired.

The matrix multiplication logic 962 can operate to combine matrices M and P to generate a raw cell-by-phenotype matrix 964 (CbPh_{raw}), where each cell of matrix 964 will indicate whether each cell expresses or lacks each phenotype. This matrix multiplication operation to compute matrix 964 can be computed as M·P^{T}.

If a practitioner does not want to exclude rare phenotypes, then averaging logic 968 can be used to average each column of matrix 964 to compute a cell expression frequency for each phenotype, which can be used to populate the combinatoric matrix 920.

However, in embodiments where a practitioner wants to exclude rare phenotypes from the combinatoric matrix 920, the GPU 960 can also employ a rare phenotype exclusion filter 966 in combination with the averaging logic 968. The rare phenotype exclusion filter 966 can use prime factorization to exclude rare phenotypes and their derivatives using an iterative process that progressively advances from the shortest phenotypes through the longest phenotypes. FIG. 9F shows an example process flow for carrying out filter 966 in combination with averaging logic 968.

To support these operations, each protein marker can be encoded by assigning each protein marker a unique prime number (e.g., CD3+=3; CD4+=7, etc.). The unique prime number that is used to encode each protein marker can be called a marker factor. With this approach, each phenotype can then be encoded by the product of its component marker factors (e.g., the phenotype CD3+CD4+ can be encoded as 3*7=21). These products can be referred to as phenotype encodings, and the phenotype encoding that has been mapped to each phenotype can be maintained in a phenotype encodings table 990 as shown by FIG. 9F. This encoding allows the filter 966 to efficiently determine which phenotypes should be excluded as rare phenotypes as explained below.

Furthermore, to support the operations of FIG. 9F, a “Do Not Evaluate” list 992 can be populated with the phenotype encodings of phenotypes that are to be excluded from matrix 920 because the process flow determines that they qualify as rare phenotypes.

The process flow of FIG. 9F can evaluate the phenotypes in loops corresponding to different phenotype lengths, starting from the shortest phenotypes. Further still, the process flow of FIG. 9F can also run separately (or in parallel) for each different patient of the single cell data 200 if multiple patients are present. At step 970, the process flow initializes to the shortest phenotype length (by setting phenotype length L equal to 1, which means that the first loop of the process flow of FIG. 9F will operate on phenotypes that are made up of a single protein marker).

At step 972, a phenotype of length L is selected from the raw cell-by-phenotype matrix (CbPh_{raw}). This phenotype will serve as the subject phenotype for steps 974, 976, 978, 980, and 982.

At step 974, the GPU determines whether the phenotype encoding for the subject phenotype (see table 990) will evenly divide (modulus zero) by any of the phenotype encodings on the Do Not Evaluate list 992. It should be understood that during the first loop through the process flow, the Do Not Evaluate list 992 will be empty, in which case step 974 will either not be performed or will always result in branching to step 976. Step 974 will play a role in the process flow during subsequent loops when longer phenotypes are evaluated.

At step 976, the GPU counts the number of expressing cells for the subject phenotype in the raw cell-by-phenotype matrix. If a Boolean gating approach is used to evaluate protein expression, step 976 can operate to sum the values in the relevant column of the raw cell-by-protein matrix which corresponds to the subject phenotype.

At step 978, the count generated at step 976 is compared with the minimum threshold that is used to decide whether a phenotype qualifies as rare.

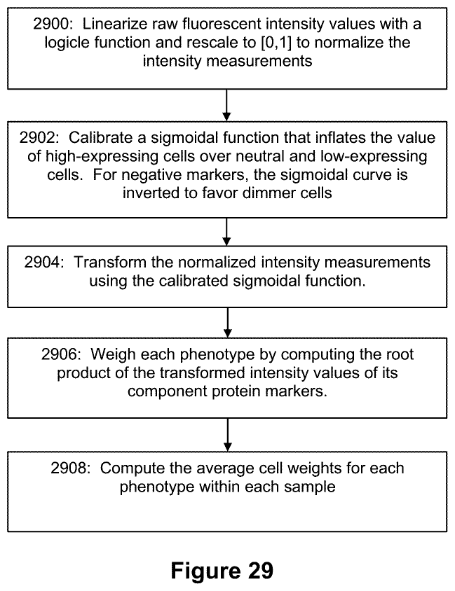

If the count for the subject phenotype passes the comparison at step 978 (e.g., the count is greater than or equal to the minimum threshold), then the process flow can proceed to step 980. At step 980, the subject phenotype is kept in matrix 920, and its cell expression frequency in the subject patient is computed (e.g., the count divided by the total number of cells in the single cell data 200 for the subject patient). The matrix 920 can be populated with this computed cell expression frequency for the subject patient and the subject phenotype. Also, it should be understood that in embodiments where a non-gating approach to evaluating protein expression is used, step 980 can operate to compute the cell values based on the non-gated values indicative of phenotype expression (e.g., see FIG. 29 discussed in greater detail below).

If the count for the subject phenotype fails the comparison at step 978 (e.g., the count is less than the minimum threshold), then this indicates that the subject phenotype qualifies as rare and the process flow can proceed to step 982. At step 982, the subject phenotype and its derivatives will be excluded from the matrix 920 by adding the encoding for the subject phenotype to the Do Not Evaluate list 992. The derivatives of a subject phenotype include all other phenotypes which include that subject phenotype; so if CD4−CD8− qualifies as a rare phenotype, this means that longer phenotypes which contain CD4−CD8− will also be excluded from the matrix 920.

At step 984, the process flow determines whether there are any remaining phenotypes of length L that still need to be considered. If yes, the process flow loops back to step 974 for the next phenotype of length L (see 986). If no, the process flow loops back to step 972 to progress to the next loop by incrementing the next larger phenotype length (L=L+1) (see 988).

Thus, on subsequent loops where the phenotypes under consideration are formed by combinations of two or more protein markers, it may be the case that the Do Not Evaluate list 992 has been populated with one or more phenotype encodings. Thus, when step 974 is reached during subsequent loops, a check can be made regarding whether a subject phenotype can be excluded from further analysis because it is a derivative of a rare phenotype (and thus also should be deemed rare). This can be efficiently accomplished using the prime factorization discussed above.

For example, consider a case where CD4− is mapped to the prime number 9, CD8− is mapped to the prime number 11, and CD95+ is mapped to the prime number 13. This would result in the phenotype CD4−CD8− being encoded as 9*11=99 and the phenotype CD4−CD8−CD95+ being encoded as 9*11*13=1287. Now, suppose that during the first loop of the FIG. 9F process flow, all of the phenotypes CD4−, CD8−, and CD95+ are deemed non-rare; but during the second loop of the FIG. 9F process flow, the phenotype CD4−CD8− is deemed rare. In this circumstance, the encoding of 99 is added to the Do Not Evaluate list 992. During the third loop, step 974 would result in a conclusion that the phenotype CD4−CD8−CD95+ should not be evaluated and excluded as rare because its mapped encoding of 1287 is evenly divisible (modulus zero) by the excluded encoding 99 (where 1287% 99=0). As such, it should be understood that the prime factorization technique used herein provided the GPU with a fast and computationally-efficient mechanism for quickly determining that derivatives of shorter excluded phenotypes should also be excluded.

Accordingly, not only does the use of a GPU offload allow the system to reduce the latency by which the single cell data 200 is transformed into the network model 210 of related phenotypes; but the prime factorization techniques described herein that can be carried out by the GPU allows the system to maintain phenotypes as compact numerical encodings that leverage the GPU's fast arithmetic operations while avoiding costly memory transfers and type conversions. These innovations allow the system to filter out low-abundance phenotypes on a GPU chip, which drastically reduces memory consumption and processing times.

While the examples of FIGS. 9E and 9F show the offloading of various operations to a GPU 960, it should be understood that these processing operations could be offloaded to other highly parallelized compute resources such as one or more field programmable gate arrays (FPGAs) and/or application-specific integrated circuits (ASICs) if desired by a practitioner.

### Example Bioinformatics System Architecture

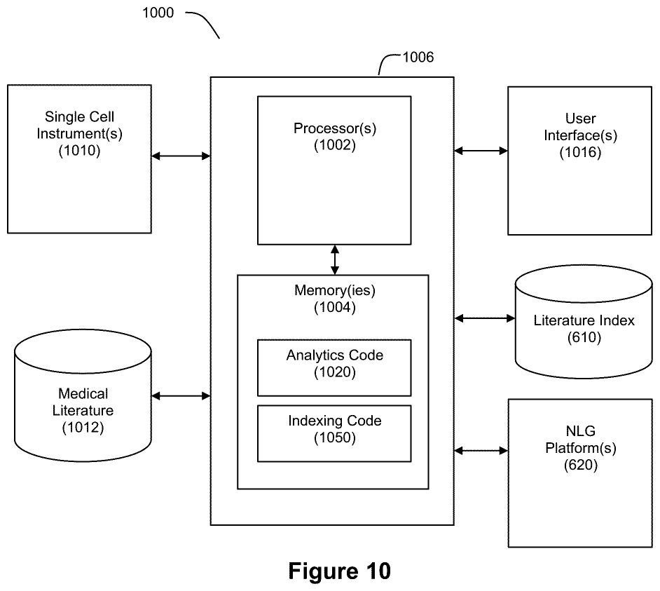

FIG. 10 shows an example bioinformatics system or platform 1000 that can be configured to carry out the transformation of single cell data 200 into insights about the single cell data 200 using techniques as discussed herein. The system 1000 may comprise one or more computer systems 1006, where the computer system(s) 1006 may comprise one or more processors 1002 with associated memory (or memories) 1004. The memory(ies) 1004 may comprise analytics code 1020 and indexing code 1050, which can be embodied as machine-readable code such as a plurality of instructions that are resident on a non-transitory computer-readable storage medium (e.g., computer memory) for execution by one or more processors to carry out the operations described herein. The bioinformatics system 1000 may also comprise single cell instrument(s) 1010 (e.g., flow cytometry instrument(s)) which can generate the single cell data 200 to be processed using the techniques described herein. One or more user interfaces 1016 can be displayed on one or more screens for receiving inputs from users and presenting outputs to users. Moreover, the bioinformatics system 1000 may have access to one or more databases which store medical literature (see 1012) and operate to generate one or more data structures 220 such as one or more literature indexes 610. The literature index(es) 610 can be housed in one or more databases. The system 1000 may also include one or more NLG platforms 620 that produce natural language outputs that synthesize data about phenotype(s) of interest from the network model 210 and data from the phenotype-related medical papers accessed via index 610.

It should be understood that the links between the computer system(s) 1006 and other components of system 1000 (e.g., the medical literature 1012, single cell instrument(s) 1010), etc.) can be any type of communication links that support data transfers as discussed herein, including but not limited to wired and/or wireless links. Moreover, such wired and/or wireless links can be implemented through one or more communication networks such as local area networks (LANs), wide area networks (WANs), the Internet, cellular networks, etc. The processor(s) 1002 can be any type of compute resource that is capable of carrying out the operations described herein. For example, the processor(s) 1002 may comprise one or more CPUs. In some embodiments, a practitioner may choose to employ highly parallelized compute resources such as one or more GPUs as processor(s) 1002 in order to improve performance and reduce the latency by which single cell data 200 can be translated into insights. For example, GPUs can be used to perform matrix manipulation operations in parallel as discussed above. As another example, a practitioner may choose to employ hybrid types of processors such as one or more CPUs in combination with one or more GPUs, where the GPU(s) can offload a variety of processing tasks from the CPU(s).

Further still, it should be understood that the architecture of FIG. 10 is an example only and variations in architecture could be employed if desired by a practitioner. For example, the computer system(s) 1006 could include multiple servers, such as a first server that runs the analytics code 1010 and a second server that runs the indexing code 1050. Thus, it should be understood that different processors 1002 can be employed to carry out different processing tasks that are described herein. As another example, the NLG platform(s) 620 may be omitted in embodiments where the insights are presented visually such as through graphical depictions of the network model 210.

In operation, a user who is performing single cell experiments may begin by selecting protein markers for measurement by the single cell instrument(s) 1010 (e.g., a flow cytometer). Some proteins, such as T and B cell markers (CD3 and CD19), are used in almost every flow experiment. Other markers can be based on the user's specific research interests. The single cell instrument(s) 1010 can then generate the single cell data 200 that encompasses the selected protein markers. For example, a flow cytometer can generate FCS files that include measurements for the user-specified protein markers. This single cell data 200 (e.g. FCS files) can then be ingested by computer system(s) 1006 for analysis.

### Examples of Analytics Code

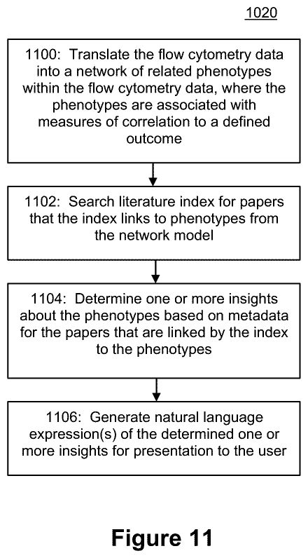

The analytics code 1020 shown by FIG. 10 can be configured to carry out the analytics operations described herein. For example, the analytics code 1020 can be configured to carry out steps 202-206 of FIG. 2 (e.g., the analytics code 1020 could be configured to perform the process flows of FIGS. 6 and/or 8C). FIG. 11 shows another example of a process flow that can be carried out by analytics code 1020 in an example embodiment. At step 1100 of FIG. 11, single cell data 200 such as flow cytometry data is translated by the analytics code 1020 into a network model 210 as discussed above with respect to step 202 of FIG. 2. At step 1102, the analytics code 1020 is executed to query the literature index 610 for papers that the index 610 links to phenotypes from the network model 210. As discussed above, this operation may be targeted to phenotypes of interest (e.g., see FIG. 5A-5C as examples) or can be generalized to all (or many) of the phenotypes of the network model 210. At step 1104, the analytics code 1020 determines one or more insights about one or more phenotypes of the network model 210 based on metadata for the medical papers that are linked by the index 610 to the one or more phenotypes. At step 1106, the analytics code 1020 generates natural language expression(s) of the determined one or more insights for presentation to a user. For example, step 1104 may identify which phenotypes are already highly reported in the medical literature and notify the user about this information via step 1106. The user can then further evaluate whether new information about the phenotype that is learned from the single cell experimentation should be published. As another example, step 1104 may result in an insight that a strongly correlated phenotype is little reported in the medical literature, which can then be reported to the user via step 1106. Once again, this reporting may provide a basis for publication or further experimentation. As yet another example, step 1104 may result in an insight that a strongly correlated phenotype is highly reported in the medical literature (including its correlation to the subject defined outcome), and this information can be reported to the user via step 1106; which may help guide the user to avoid further research into an area that is already well covered or otherwise confirm a diagnosis that a user believes may be warranted.

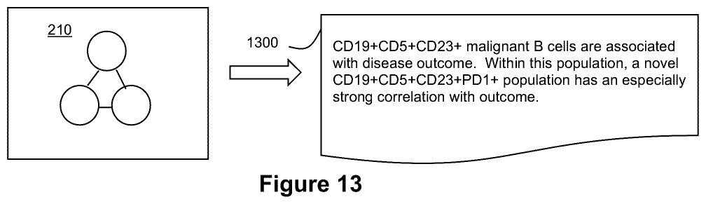

FIG. 12 shows an example of a network model 210 that has been augmented with phenotype-specific medical literature information and phenotype-specific insights that can be created using the techniques described herein. Thus, the nodes 402 shown by FIG. 12 have been augmented with metadata about the nodes' corresponding phenotypes such as correlations derived from the single cell data 200 and insights derived from relevant medical literature. Examples of metadata linked to various nodes 1202, 1204, and 1206 are shown as 1212, 1214, and 1216 respectively. This metadata 1212, 1214, and 1216 can identify the correlation measure, number of connected papers, and one or more summarized insights for the phenotype applicable to the subject node 1202, 1204, and 1206. It should be understood that the paper counts and summarized insights shown by FIG. 12 are hypothetical and for purposes of explanation only. In this example, FIG. 12 shows node 1202 as a local “hot spot” in the network model 210 where CD19+CD15+CD23+ cells are moderately correlated with outcome (Cohen's d=1.7) and well represented in the literature (see 1212). Their function is clearly understood, and the phenotype is mentioned by name in at least 173 papers. Meanwhile, CD19+CD15+CD23+PD1+ cells are strongly correlated with outcome (Cohen's d=2.9) but poorly understood (see node 1204). Only two papers report the same finding without providing much explanation. The system can report these two phenotypes as one set. For example, steps 604, 606, and 608 of FIGS. 6 and/or 8C can produce a natural language output such as “CD19+CD5+CD23+ malignant B cells are associated with disease outcome. Within this population, a novel CD19+CD5+CD23+PD1+ population has an especially strong correlation with outcome.” FIG. 13 shows a sample output report 1300 in this regard, which can be generated by providing selected phenotypes and associated metadata derived from the network model 210 to an NLG platform 620 as per the process flows of FIGS. 6 and/or 8C as discussed above.

Further still, some practitioners may find it desirable to produce a visualization of an augmented network model 210 as shown by FIG. 12 as the determined insight that is presented to a user via step 206. With such a visualization, disease-associated phenotypes in some neighborhoods of the network model's network structure will overlap with established biology from literature. These “light zones” represent well-established phenotypes. In other neighborhoods, disease-associated phenotypes will not overlap with established biology, or at least not with any biology from the domain covered by the literature index 610. These “dark zones” represent unique opportunities to make a new discovery or draw connections across disparate areas of research. Scientists can interact with the network model 210 of FIG. 12 to better understand how their data aligns with current biomedical knowledge.

### Examples of Indexing Code

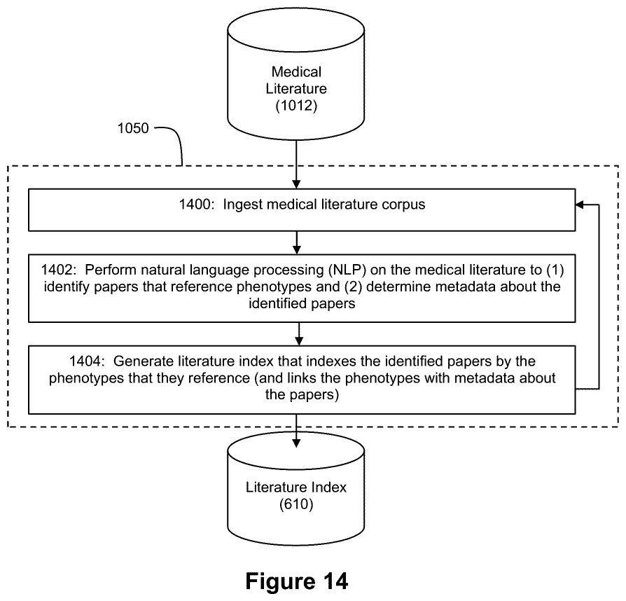

The indexing code 1050 shown by FIG. 10 can be configured to carry out creation of the data structure 220 described herein. FIG. 14 shows an example process flow for creating the data structure 220. At step 1400, the indexing code 1050 ingests a medical literature corpus from data source(s) 1012. At step 1402, the indexing code 1050 performs natural language processing (NLP) on the medical papers of the ingested medical literature corpus to (1) identify papers that reference protein markers and protein marker combinations so that a conclusion can be drawn about whether the papers reference phenotypes from the network model 210 and (2) determine metadata about the identified medical papers. This metadata can take the form of metadata 616 as discussed above. At step 1404, the indexing code 1050 generates literature index 610, where the literature index 610 indexes the identified papers by the phenotypes that they reference and links these phenotypes with the metadata about those papers.

The medical papers that are processed at steps 1400 and 1402 are expected to comprise large amounts of unstructured text. Parsing unstructured text is inherently challenging, and extracting cell type information from such unstructured text is particularly difficult. First, the same phenotypes can be described multiple ways in different papers, as in “bright for HLA-DR and CD4”, “HLA-DR/CD4 co-expression”, or “HLADR+CD4+”. Second, extracting cell type information requires specific knowledge of flow cytometry. Traditionally, only a domain expert would know that HLA-DR+ is equivalent to HLADR+, not HLA− and DR+. Finally, each protein can have multiple names, as in CCR7 and CD197. Other common markers, such as CD3, aren't proteins at all but are actually multiprotein complexes that include CD3a. The NLP that gets performed on the unstructured text as part of the indexing code 1050 can be designed to capture all of these cases and index them under the same machine-readable label.

FIG. 15 shows another example process flow for indexing code 1050. As an example, this process flow can include operations such as the following.

At step 1500, the system can download full-text papers from sources such as PMC Open Access Subset and/or bioRxiv. The papers can be downloaded as full-text XML data. In an example where the system is being used to process flow cytometry data, the corpus of papers accessed at step 1500 can be all papers with the keyword “flow cytometry”.

At step 1502, the system can perform a string search for common delimiters that may be present in the medical papers that would indicate that a medical paper references a protein marker. Examples of delimiters that can be used for this search include:
+, pos, high, hi, bright
−, neg, low, lo, dim, dull, null
int, het, mid

This can yield a set of papers (or subsets within papers such as excerpts (e.g., paragraphs)) which use terms relevant to protein markers. It should be noted that the searching can search for combinations of these keywords such as multiple instances of such keywords in the same sentence of a paper if desired by a practitioner.

At step 1504, a fine-tuned LLM can be used to convert the text of the medical papers into a machine-readable data array (e.g., a machine-readable JSON array). For example, a natural language interface such a chatbot interface (e.g., ChatGPT such as OpenAI's ChatGPT4 API) can be used to extract phenotypes from selected passages identified as a result of step 1502.

At step 1506, the system can use a grounding tool such as Gilda (see Gori, Benjamin M., “Gilda: biomedical entity text normalization with machine-learned disambiguation as a service”, Bioinformatics Advances, Vol. 2, Issue 1 (2022)) to map protein names for the extracted protein phenotypes to their unique UniProt IDs. This process can disambiguate references that may be found in literature that are describing the same protein marker but are using different terms to do so.

At step 1508, the system can store the extracted phenotypes and their associated data as machine-readable JSON strings.

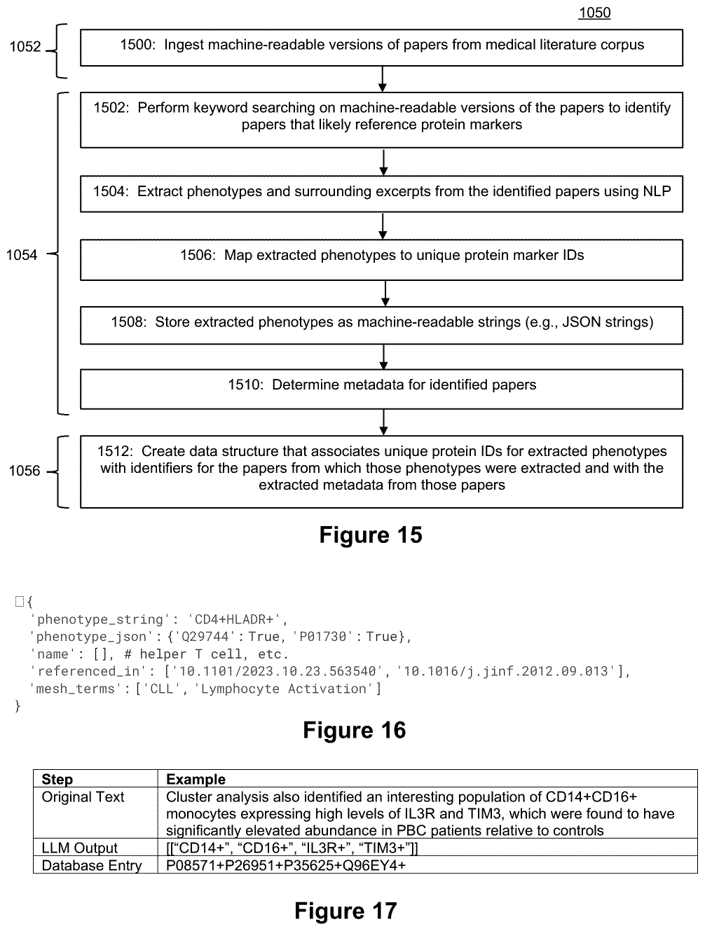

At step 1510, the system can link the extracted phenotypes to each paper's keywords, which can include author-provided keywords and MeSH terms assigned by manual reviewers. This operates to store phenotypes in a linked database that includes metadata such as digital object identifier (DOI) number, publication date, keywords, etc. An example of a JSON string for an extracted phenotype and associated metadata is shown by FIG. 16. It should be understood that FIG. 16 is hypothetical and presented for the purpose of explanation with fictional DOIs.

The medical literature index 610 can be generated by creating a data structure that associates protein IDs for the extracted phenotypes with identifiers for the papers from which those phenotypes were extracted and with the metadata extracted from those papers (see 1512 in FIG. 15).

FIG. 17 shows an example of how unstructured text can be translated into an extracted set of protein markers (which define a phenotype) as a result of step 1504 and then further translated into a database entry in the index 610 at step 1512.

### Example Multi-Agent Bioinformatics System

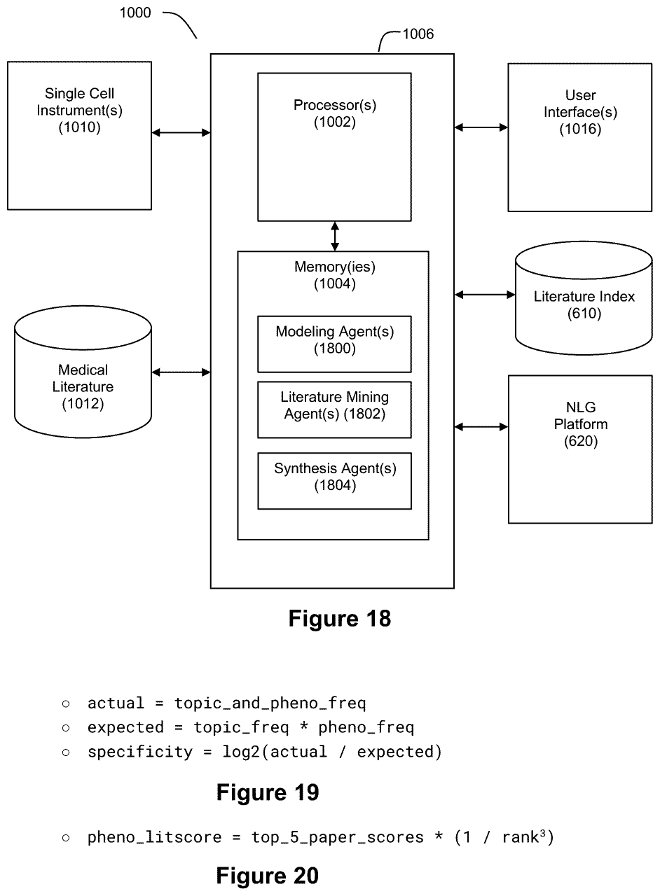

The bioinformatics system 1000 can also be configured as a multi-agent system; an example of which is shown by FIG. 18. With the example of FIG. 18, the bioinformatics system 1000 includes a modeling agent 1800, a literature mining agent 1802, and a synthesis agent 1804. These agents 1800, 1802, and 1804 can be embodied by code or software resident on a non-transitory computer-readable storage medium and that is executable by the one or more processor(s) 1002 of the bioinformatics system 1000. Each agent 1800, 1802, and 1804 is capable of being executed concurrently and independently with each other. This allows for coordinated operations by different agents while also being capable of frequently updating the data structure 220 with new literature as time goes on. This ability to leverage a distributed computing architecture to update different aspects of the system provides the system 1000 with powerful scalability.

Underlying these agents 1800, 1802, and 1804 is the network structure of the network model 210 that allows for efficient and contextual coordinated analysis of single cell data 200 and relevant medical literature.

Moreover, it should be understood that the system can employ a plurality of modeling agents 1802, literature mining agents 1804, and/or synthesis agents 1804 if desired by a practitioner. The computer system 1006 can be a distributed computer system where different servers or other computing platforms are capable of executing one or more different agents 1800, 1802, and 1804 concurrently with each other so that multiple sets of single cell data 200, multiple network models 210, multiple literature indexes 610 (or a common literature index 610 can be updated by different mining agents 1804), etc. can be created and/or updated in a distributed fashion for improved computing performance. Moreover, different synthesis agents 1804 can be employed that are capable of carrying out different types of synthesis operations. For example, synthesis agent A may be configured to produce visualizations of the network models 210 (which may include network models 210 that have been augmented with phenotype-relevant medical literature data), while synthesis agent B may be configured to produce natural language summarizations of medical literature that is relevant to one or more identified phenotypes of interest, while synthesis agent C is configured to carry out still another synthesis operation, etc.

Further still, the mining agent(s) 1804 can operate over time to update the network model 210 with metadata derived from new medical papers that have been recently published or recently discovered. In this fashion, the network model 210 can evolve over time so that single cell data 200 created at Time X and be transformed into a network model 210 that effectively learns new insights derived from medical literature 1012 as new knowledge gets added to the medical literature 1012 over time (Time X+Y in the future). Accordingly, users can re-visit their single cell data 200 via the system 1000 to potentially learn new things about their single cell data 200 as new knowledge gets added to the medical literature 1012.

Further still, the system can operate on multiple sets of single cell data 200 over time that can be aggregated together in a network model of related phenotypes that spans multiple experiments. Each experiment would be linked to a set of single cell data 200 and can be linked to relevant medical literature, ontological terms, and insights as discussed herein. This can allow researchers to evaluate whether outcomes such as disease associations and insights derived from the single cell data are consistent across different experiments. For example, the mining agent(s) 1804 can also operate to add user-specific entries to the medical literature database/index to link entries in the medical literature database/index so that a group of the top disease-associated phenotypes (e.g. Top 10) as derived from multiple experiments can be linked by the database to user-provided topics of interest.

The modeling agent 1800 is configured to translate single cell data 200 into the network model 210 using the techniques discussed herein. For example, the modeling agent 1800 can be configured to carry out step 202 of FIG. 2. As an example, the modeling agent 1800 can perform the FIG. 9A process flow to produce the network model 210 from single cell data 200.

The literature mining agent 1802 is configured to mine the medical literature 1012 to create a literature index 610 that can be integrated, mapped, or otherwise linked into the network model 210. For example, the literature mining agent 1802 can be configured to carry out step 204 of FIG. 2. As an example, the literature mining agent 1802 can be configured to perform the FIG. 14 and/or FIG. 15 process flows to produce the literature index 610. The literature mining agent 1802 may also be configured to carry out step 800 (see FIGS. 8A and 8C).

The synthesis agent 1804 is configured to synthesize the single cell data 200 as embodied by the network model 210 produced by the modeling agent 1800 in combination with phenotype-relevant medical papers as identified by the mining agent 1802. For example, the synthesis agent 1804 can be configured to carry out step 206 of FIG. 2. As an example, the synthesis agent 1804 can be configured to perform the process flows of any of FIGS. 5A-5C. The synthesis agent 1804 may also be configured to carry out steps 600, 604, 606, and 608 of FIGS. 6 and 8C.

Accordingly, the synthesis agent 1804 is capable of synthesizing the results of the network modeling and the relevant literature mining into broader patterns. To help support such synthesis between single cell data 200 and relevant medical literature, the mining agent 1802 and/or the synthesis agent 1804 can be configured to compute scores for phenotype-relevant medical papers that will quantify one or more characteristics of the phenotype-relevant medical papers and support techniques for ranking phenotypes based on their statistical and ontological significance.

Combinatoric enumeration of phenotypes from single cell data 200 as discussed herein can produce an overwhelming number of disease-associated phenotypes. Accordingly, there is a need for an intelligent way to rank populations based on their statistical and ontological significance. The system 1000 can consider a number of factors to do this.

For example, the system 1000 can consider the phenotype itself and its relationship to the user's data. This can involve two factors:
Disease association. The synthesis agent 1804 can consider how well each phenotype correlates with patient outcome (e.g., treatment response). This can be done using any zero-centered measure of effect size such as Pearson's correlation coefficient or Cohen's d. As noted above, this information can be included in the network model 210 of related phenotypes.
Simplicity. As noted above, the system can penalize effect size based on the number of protein markers in the phenotype. The penalty factor favors simpler phenotypes that are easier to interpret and more likely to validate to new data. To provide penalties for longer phenotypes (or rewards for simpler phenotypes), a weighted correlation score can be computed as discussed above.

Also, for each instance of a phenotype in the literature, the system can evaluate how strongly related (e.g., notable) each phenotype-related medical paper is to that phenotype.
Notability. The literature mining agent 1802 and/or synthesis agent 1804 can compute a notability score for each medical paper that is linked to a phenotype. The notability score for a medical paper can indicate how strongly a subject phenotype may relate to the focus or important aspects of that medical paper. As an example, the literature mining agent 1802 can compute each paper's notability score as that paper is mined from medical literature 1012 and added to the literature index 610 (for example, the medical paper metadata 616 in the literature index 610 can include each paper's notability score). For each paper, the notability scoring can consider whether the subject phenotype was mentioned in a high-visibility section of the phenotype-linked medical paper (like the title or abstract) or a low-visibility section of the phenotype-linked medical paper (like the supplemental or methods). An example scoring table for the notability score by paper section is shown in the table below.

| Section | Score |
|---|---|
| Title | 5 |
| Abstract | 4 |
| Introduction | 2 |
| Methods | 1 |
| Results | 2 |
| Discussion | 3 |
| Supplementary | 0 |

As an example, a paper's notability score can be computed using its highest-scoring section (e.g., if a paper includes the phenotype in its title, it score would be 5 (where 5 would be the maximum score). However, some practitioners may choose to compute the notability score using other techniques such as by aggregating the scores for applicable sections (e.g., if a paper includes the phenotype in its abstract and introduction, but no other sections, its notability score could be 6). It should also be understood that this scoring table is an example only, and a practitioner may choose to employ other point values for different paper sections and/or other score scaling techniques.

Also, for each instance of a phenotype in the literature, the system can consider how the literature and phenotypes are related to a topic of interest. In this regard, the system can compute relevance scores. The topic of interest can be user-specified, and the user may specify the topic of interest as an input for the system 1000 via user interface 1016.
Relevance. The literature mining agent 1802 and/or synthesis agent 1804 can also compute a relevance score for each medical paper that is linked to a phenotype. The relevance score for a medical paper can indicate how closely related the medical paper is to the topic of interest. For each paper, the relevance scoring can consider whether the paper relates to the topic specified by the user using NLP techniques. For example, if the user was interested in COVID-19, the relevance scoring could also consider additional topics that are related to COVID-19 such as pneumonia or respiratory disease. In an example relevance scoring approach, the topic of interest for the user can serve as the target term for scoring. The topic(s) addressed by the medical paper can be defined by the Medical Subject Heading (MeSH) term(s) that are associated with the medical paper. As such, it should be understood that the literature index 610 can also index papers by the topics they address (e.g., the MeSH terms applicable to the papers can be included in the medical paper metadata 616). If a paper does not have MeSH terms, NLP techniques such as open-source machine learning (ML) tools (e.g., WellcomeBertMesh) can be used to predict MeSH terms for a paper. The relevance scoring can score a paper based on the number of hops that are needed to progress from the paper's MeSH topic to the user's target term via a MeSH ontology tree (where fewer hops would translate to higher scores). For example, the relevance score can be computed as Relevance=5−#hops, where #hops is a value that represents how many hops are required to traverse the MeSH ontology tree from the paper's MeSH term to the user's target term. However, it should be understood that this scoring model is only an example, and a practitioner may choose to employ different scaling or values for computing the relevance score.

Also, the system can consider the specificity of the relationship between the phenotype and user-direct topic of interest according to relevant medical literature.
Specificity. The literature mining agent 1802 and/or synthesis agent 1804 can also compute a specificity score that indicates how specific a phenotype is to the user's topic of interest. As such, the specificity score considers how specific a phenotype is to a given context. For example, CD3⁺CD4⁺ is a well-known phenotype (helper T cells) that shows up in almost every immunology paper. CD19⁺CD5⁺CD25⁺ is a rare population that is almost exclusively observed in B-ALL patients. The specificity scoring can prioritize phenotypes that are unique to the user's target domain. As such, the specificity score for a phenotype can tell a user if a phenotype is uniquely linked to the topic of interest or if the phenotype may just be popular across all literature (e.g., a quantification of a relationship between how many medical papers mention both the phenotype of interest and the topic of interest versus how many medical papers mention the phenotype of interest but do not address the topic of interest). FIG. 19 depicts an example scoring approach for computing specificity scores with respect to phenotypes and a topic of interest, where an approach such as a Jaccard similarity score is used. In this example, the “actual” component can be computed as the number of papers that mention the topic of interest AND the phenotype of interest. Thus, if a given paper mentions both the topic of interest and the phenotype of interest, then it would be included in the “actual” count. The “expected” component can be computed as the product of (1) the number of papers that mention the topic of interest and (2) the number of papers that mention the phenotype of interest. Specificity can then be computed as the ratio between actual and expected paper counts, as shown by FIG. 19. However, it should be understood that other techniques could be used for computing specificity scores if desired by a practitioner.

Further still, the literature mining agent 1802 and/or synthesis agent 1804 can also compute aggregated or composite scores based on the scores relating to the phenotypes, medical papers, and topics of interest.

For example, notability and relevance can each be scored on a scale of 0 to 5 and added together to produce an aggregate paper score applicable to a phenotype of interest. Paper scores can then aggregated across the top five-scoring papers, with additional emphasis placed on the single top-scoring paper—e.g., see FIG. 20. For example, with this approach, each paper's weight in the aggregated score can be computed as a function of the score ranks for the papers so that the top-scoring paper gets the largest weight, the bottom-scoring paper gets the smallest weight, and the middle-scoring papers get weights between the largest and smallest weights.

Also, specificity can be rescaled to 0 to 5 and added to the aggregate paper score to produce a literature score (litscore). The litscore can thus be scaled 0 to 15 and describes the relationship between a phenotype and the user-described topic-of-interest. For example, a high-scoring phenotype might appear in the title of paper about COVID-19, as in “Immunophenotyping characteristics of COVID-19 patients: Peripheral blood CD8+HLA-DR+ T cells as a biomarker for mortality outcome”. Moreover, a high-scoring phenotype will typically not appear in any (or not as many) papers that are not about COVID-19.

Finally, the litscore can be added to the disease association score defined earlier (e.g., weighted correlation). In an example approach, disease association can be rescaled to [0, 15] to provide equal weight to the litscore. This value can be adjusted to emphasize either highly-correlated or highly-interpretable phenotypes.

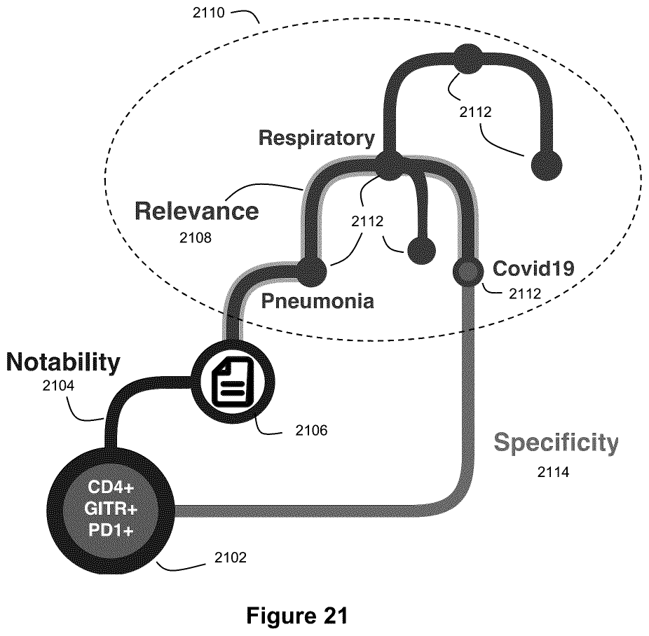

Thus, it should be understood that the bioinformatics system 1000 can score each occurrence of a phenotype in medical literature using the techniques described herein. This process can be represented as a graph consisting of phenotypes, papers, and MeSH ontologies (see FIG. 21). In the example graph of FIG. 21, the edges represent co-occurrences or relationships between these entities, and are weighted using the scores defined above (notability, relevance, and specificity). The litscore is the summation of these weights and represents the semantic closeness between each phenotype and the user-defined topic of interest. For example, FIG. 21 shows that a phenotype node 2102 from the network model (which can be weighted by its disease association and marker complexity) can be linked with a medical paper 2106 that references phenotype 2102 via a notability link 2104 that is based on the paper's notability score for the phenotype 2102. The phenotype 2102 and paper 2106 can be further connected with a medical term ontology 2110 (e.g., a MeSH hierarchy), where the ontology 2102 includes a plurality of ontological nodes 2112 that correspond to different ontological terms (e.g., MeSH terms) that are connected hierarchically based on the terms' relationships with each other. Relevance links 2108 can identify the number of hops between the paper's keyword (which is “Pneumonia” in the example of FIG. 21) and a user-defined topic of interest (which is “COVID-19” in this example). Thus, it can be seen that the relevance link 2108 can be based on the number of hops between nodes 2112 from the paper's keyword (pneumonia) and the topic of interest (COVID-19). The specificity link 2114 can be based on the specificity score relating the phenotype 2102 (CD4+GITR+PD1+ in this example) to the topic of interest (COVID-19).

While the example of FIG. 21 shows only a single paper 2106 connected to the phenotype 2102, it should be understood that the phenotype 2102 may be linked to multiple papers 2106 in situations where the phenotype 2102 has connections with multiple papers.

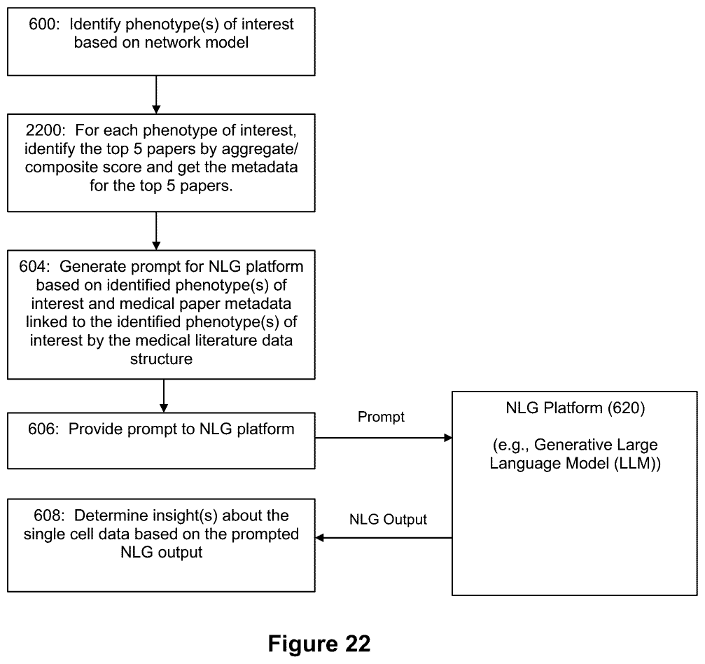

FIG. 22 shows an example process flow that can be carried out by an example synthesis agent 1804 using aggregated/composite paper scores. Steps 600, 604, 606, and 608 can be carried out at discussed above with reference to FIGS. 6 and 8C. The FIG. 22 process flow includes step 2200, where for each phenotype of interest, the top X medical papers according to their aggregated/composite scores with respect to the subject phenotype of interest are selected. In an example, the value of X can be 5 so that the top 5 papers according to aggregated/composite score are selected. However, it should be understood that other values for X could be used by a practitioner if desired (e.g., X=2, 3, 7, 10, etc.). The metadata for these top 5 papers is obtained from the literature index 610 and/or the network model 210 (if the network model 210 has been augmented with such metadata as a result of a mapping operation 800 as discussed above). As an example, the obtained paper metadata can comprise the title, abstract, and phenotype-relevant paragraphs from each top 5 paper, and step 2200 can concatenate these metadata items for each top 5 paper. This concatenated metadata can then be used to generate a prompt that will get provided to the NLG platform 620 (see FIGS. 7B and 7C). The NLG output from this prompt can be presented to the user as a summarization of medical literature that is relevant to the user's single cell data 200 (see FIG. 7D). This NLG output can be presented to the user via user interface 1016 in any of a number of fashions. For example, the summarization can be accompanied by actual excerpts from each paper, and the output report can be structured for access through the user interface 1016 via drop down sections and/or tabs on the user interface 1016.

Integration of Network Models with Medical Ontologies:

In some example embodiments, practitioners may find it desirable to link the single cell data 200 with external medical knowledge, where this external medical knowledge can comprise medical information that exists outside the single cell data 200 such as information derived from medical literature (as discussed above) and/or one or more medical ontologies. For example, practitioners may find it desirable to not only link the network model 210 of related phenotypes that are derived from the single cell data 200 to relevant medical literature but also link the network model 210 to a medical ontology.

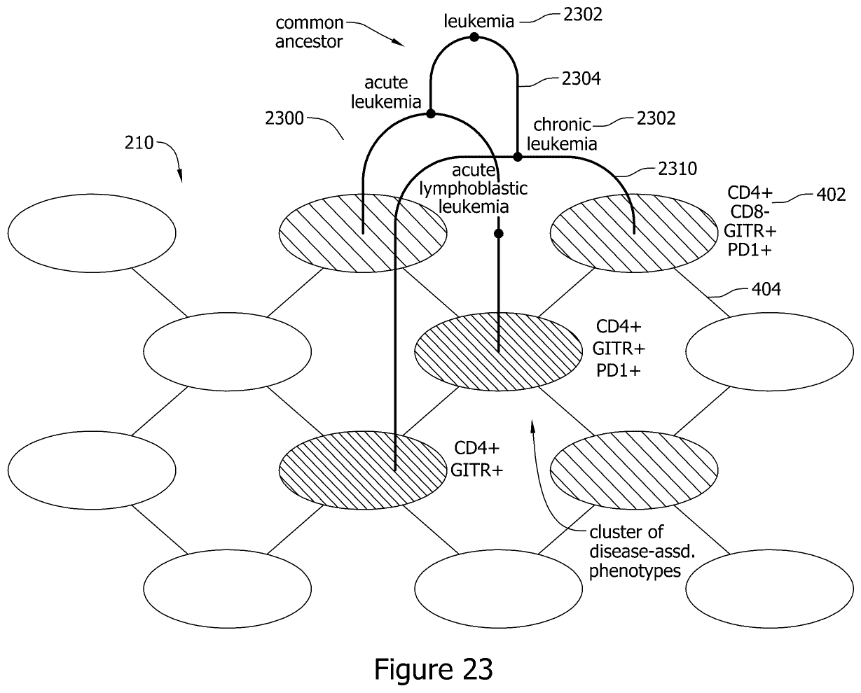

A medical ontology can be represented by a data structure whose nodes correspond to different ontological terms, where connections between the ontological nodes are based on relatedness between those ontological terms. Accordingly, such a data structure can also exhibit a network structure, and this network structure can define a hierarchical tree arrangement where nodes that are toward the root node correspond to more general terms (e.g., cancer) while nodes that are further away from the root node correspond to more specific terms (e.g., types of cancer such as leukemia, etc. and so on (e.g., specific types of leukemia, etc.)). As an example, the ontological network data structure can be pulled from an ontology such as the MeSH database where nodes in the ontological network correspond to disease classifications. FIG. 23 shows an example of such an ontological network 2300, where ontological nodes 2302 correspond to different disease classifications, and where relations between disease classifications are identified by ontological connections 2304.

The ontological network 2300 can be linked with the network model 210 as shown by FIG. 23. Through the linkage of the network model 210 with insights derived from medical literature 1012, the phenotype nodes 402 of the network model 210 can become associated with disease classifications. The associated disease classifications for the nodes 402 of the network model 210 can be derived from the relationship data values that are associated with the nodes' corresponding phenotypes and from the information derived from the phenotype-related medical papers (e.g., MeSH terms or other summarizations extracted or derived from the phenotype-related medical papers). The disease classifications that are assigned to phenotype nodes 402 of the network model 210 as a result of the linkage and automated analysis of medical literature can be used as a basis for further integrating or connecting the network model 210 with the ontological network 2300 via disease classifications in common with the ontological network 2300 (see connections 2310 between nodes 2302 of the ontological network 2300 and nodes 402 of the network model 210). This integration between the network model 210 and the ontological network 2300 provides further powerful contextualization because the associated disease classifications for the phenotype nodes 402 of the network model 210 can be placed into the context of a related ontology of disease classifications. For example, not only can a synthesis agent 1804 traverse through connections 404 to find and evaluate the relevance of related phenotypes but the synthesis agent can also traverse through connections 2310 and 2304 to contextualize phenotypes within a larger set of disease classifications. As such, synthesis agents 1804 can also use the integration between the network model 210 and ontological network 2300 as a basis for identifying and presenting insights to users. For example, the linked network model 210 and ontological network 2300 can be queried by a synthesis agent 1804 with a goal of identifying clusters of disease-associated phenotypes along with their common ontologies so that the determined insights and natural language outputs can appropriately contextualize correlation measures in the network of related phenotypes.

Thus, the ontological network 2300 can also provide the system with more powerful capabilities for phenotype annotation. Some populations are generic stress markers that show up in every disease. Other populations are highly specific to one clinical subtype. The system can use the specificity scores for the phenotypes with respect to phenotype-relevant medical literature to annotate each phenotype with the appropriate level of specificity. For example, CD3+CD4+ T-cells are commonly reported across all immune literature, so the appropriate level of specificity is “immunology”. CD19+CD5+CD25+ is almost exclusively reported in the context of B-ALL, so the appropriate level of specificity of “B-ALL”. For example, for each phenotype, the system may subset ontological nodes (topics) that coincide with the phenotype in at least N papers (e.g., N=10 papers, although it should be understood that values of N other than 10 may be used if desired by a practitioner). The system may then group topics that share a common ancestor within a predefined number of hierarchical levels (“hops”) in the ontological tree, with the threshold for common ancestry set at L hops (e.g., L<=2 levels; although it should be understood that values of the L threshold other than 2 may be used if desired by a practitioner). The highest-level ontological node in each group (i.e., the most general topic) can then be assigned as the specificity level for the phenotype. Each phenotype may have multiple topics representing disparate regions of the tree.

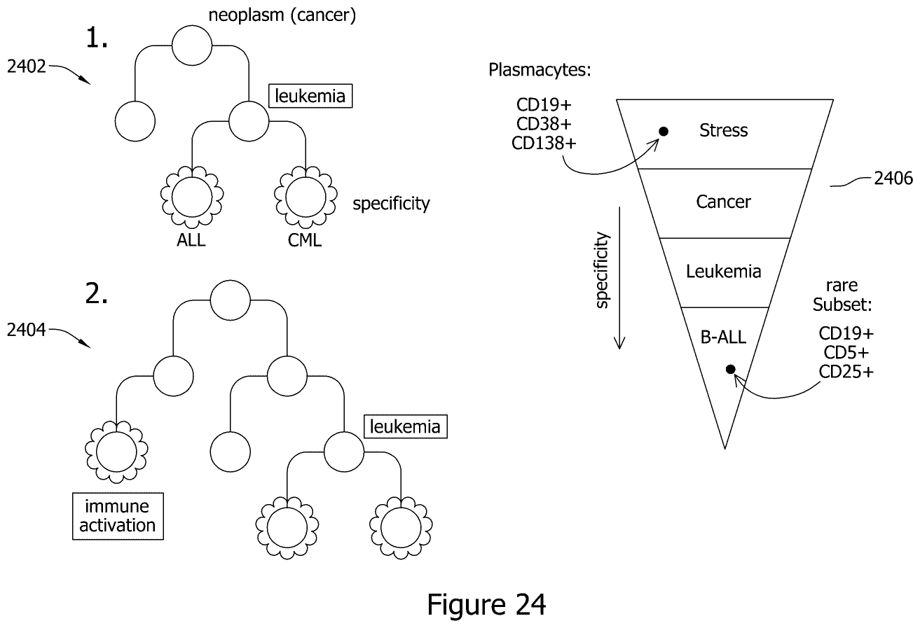

FIG. 24 shows an example of how the system 1000 can be used to annotate phenotype nodes 402 with an appropriate level of specificity. For example, with situation 2402 shown by FIG. 24, the specificity scores assigned to the phenotypes concentrate in one region of an ontological network (e.g., MeSH tree). The system 1000 can thus label the subject phenotype(s) with the root node of this example (leukemia). With situation 2404 shown by FIG. 24, the specificity scores concentrate in two regions of the ontological network (e.g., MeSH tree)—one related to disease (leukemia) and one related to function (activation). In this instance, the system 1000 can label the subject phenotype(s) with both topics from the ontological network. The inverted pyramid 2406 of FIG. 24 shows an example of how phenotypes from the network model 210 can have varying levels of specificity with respect to terms from an ontological network 2300.

In another example, a synthesis agent 1804 can use the linked network model 210/ontological network 2300 to determine further insights about phenotype organization. In addition to selecting the “best” cell types, the system 1000 can also configure a synthesis agent 1804 to use the combinatoric network 210/2300 to report related phenotypes as a set if they are directly linked to each other. For example, let's say that the network model 210 indicates that CD19⁺CD15⁺CD23⁺ B cells are moderately correlated with disease but well-represented in the literature. Their function is clearly understood and the phenotype is mentioned in at least 173 leukemia papers. Meanwhile, the network model 210 also shows that a rare child population, CD19⁺CD15⁺CD23⁺PD1⁺, is strongly correlated with outcome but poorly understood. Only two papers report the phenotype and don't offer much explanation. The system 1000 can report these two findings as a set: “CD19⁺CD15⁺CD23⁺ is a well-known biomarker of leukemia, as confirmed by this dataset. Within this population, a rare PD1-expressing is highly predictive of immunotherapy response.”

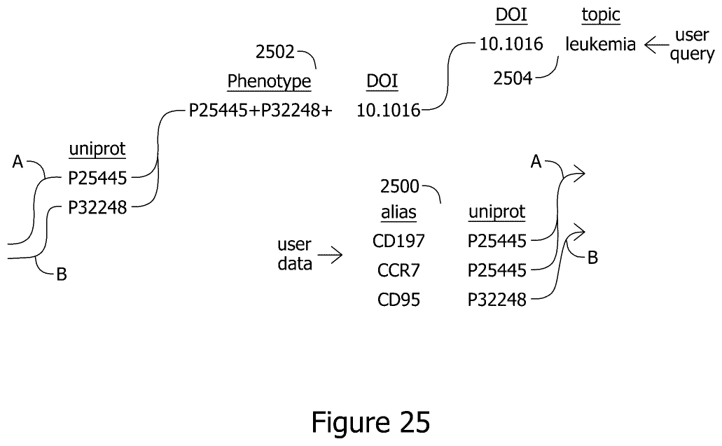

The linked network model 210 and ontological network 2300 can be further embodied as one or more databases within system 1000 where linkages can span from protein names (e.g., CD197) to an ontological term (e.g., leukemia). An example of such database linkages is shown by FIG. 25. Associations 2500 can connect protein markers from the single cell data 200 with a standardized protein identifier such as a UniProt ID. The protein marker combinations (phenotypes) can be linked with relevant medical literature (e.g., DOIs for phenotype-related medical papers) via associations 2502 using the techniques as discussed herein. Moreover, these phenotype-related medical papers can be linked to ontological terms via associations 2504. Such a database can be leveraged through system 1000 in both a feed backward and feed forward manner.

In a feed backward approach, the system can perform an unbiased search for disease-associated phenotypes and further mine medical literature to see if anyone has reported the same finding. This approach can highlight knowledge gaps that may lead to new discoveries.

In a feed forward approach, the system can take the phenotypes that are reported in a given context and replicate them in a user's single cell data 200. For example, if a user is studying leukemia, the system 1000 can identify the set of phenotypes that are reported by the medical literature in connection with leukemia and then assess which of these reports validate to the user's single cell data 200. This feed forward approach can show how results support or contradict the current state-of-the art knowledge in the field.

Because the phenotypes are stored and linked in a machine-readable database using the techniques described herein, the system 1000 can easily run feed-forward and feed-backward searches on each new single cell data set and report out best sets of disease-associated phenotypes.

### Non-Gating Approach for Evaluating Protein Marker Expression:

As noted above in connection with step 902 of FIG. 9A, some practitioners may find it desirable to employ a non-gating approach to evaluating whether protein markers are expressed by the cells of the single cell data 200. The non-gating approach described herein provides several technical benefits over conventional gating approaches where Boolean cutoffs are used by conventional gating approaches to evaluate whether protein markers are positively expressed or negatively expressed by a cell in the single cell data.

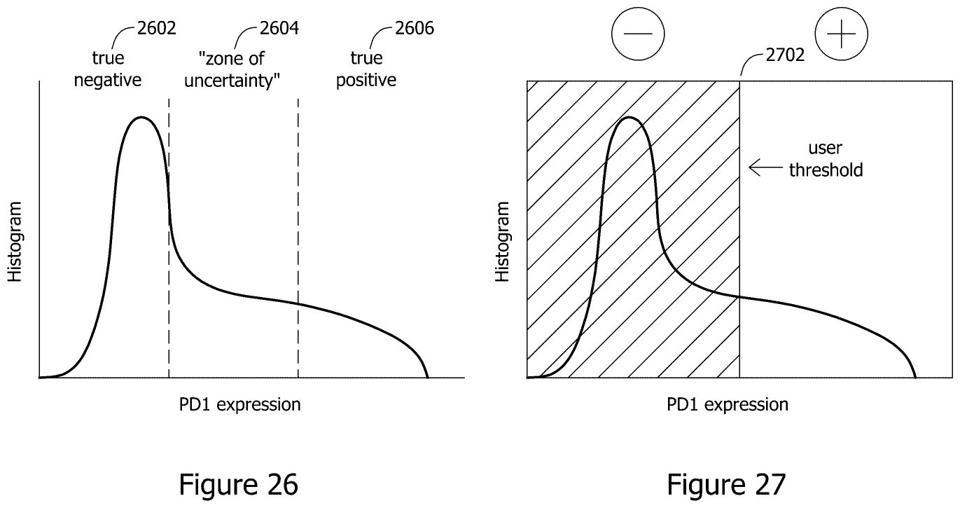

As background, flow cytometrists often describe cells as either expressing or lacking a particular protein, as in CD3+ T cells. However, defining expression and non-expression is not always straightforward. FIG. 26 shows an example histogram of PD1 expression. With reference to FIG. 26, most cytometrists would agree that cells in the first third (see 2602) are negative and cells in the last third (see 2606) are positive. However, cytometrists would draw a slightly different threshold inside the middle third (see 2604). This middle third 2604 can be referred to as a zone of uncertainty for protein expression. Accordingly, there is no consensus on what defines true PD1 expression.

Traditional workflows force scientists to choose a single fluorescent intensity cutoff (see 2702 in FIG. 27), introducing user bias into the analysis. This Boolean cutoff creates distinct zones for assigning positive and negative expression to protein markers, where the zone of uncertainty is effectively eliminated with respect to the assignment of positive expression conclusions and negative expression conclusions. This is particularly true when dealing with rare markers such as PD1 or IFNg. Because the intermediate population is relatively large (as seen by the histogram line plots in FIGS. 26 and 27), even small adjustments in gate placement can wash out true biological variation in the positive population. Thus, traditional manual gating approaches can obscure protein markers that do not conform to a strict on/off binary. For example, T-cells that secrete high levels of IFNy are functionally distinct (i.e., are polyfunctional) compared to T-cells that secrete low levels of IFNy.

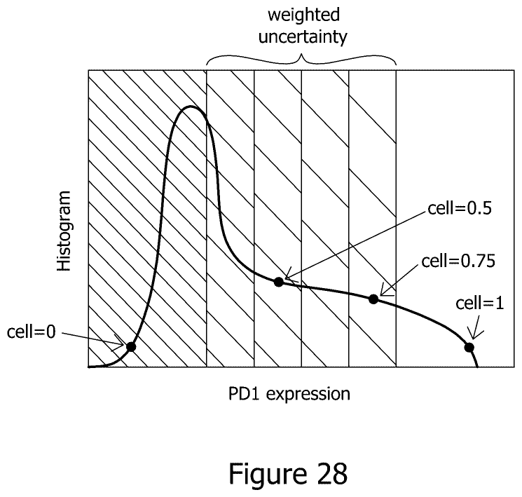

With the innovative non-gating approach described herein, the system can estimate phenotype abundance without relying on singular cutoffs. With such a non-gating approach, the system weighs cells by their relative uncertainty. The more confident we are that a cell is positive, the larger the weight. Similarly, the more confident we are that a cell is negative, the smaller the weight. An example of this is shown by FIG. 28, where cells in the true negative region (e.g., see 2602 in FIG. 26) can be weighted as zero, while cells in the true positive region (e.g., see 2606 in FIG. 26) can be weighted as 1. However, cells in the zone of uncertainty (e.g., see 2604 in FIG. 26) are assigned weights that capture more granularity in protein expression (such as cells with weights of 0.5 that are toward the middle of the zone of uncertainty and cells with weights of 0.75 toward the upper end of the zone of uncertainty). The non-gating approach effectively de-prioritizes ambiguous cells over clear positives, allowing us to approximate traditional gates without relying on a singular cutoff.

FIG. 29 depicts an exemplary process flow for carrying out step 902 of FIG. 9A using a non-gating approach.

At step 2902, the system linearizes the raw fluorescent intensity values of the single cell data 200 using a logical transformation function, and the linearized values are re-scaled to [0,1]. This operates to normalize the expression of each protein marker. The intensity values can be represented as I, and the normalized intensity values can be represented as I′.

At step 2904, a sigmoidal function is calibrated so that the sigmoidal function will inflate the value of high-expressing cells over neutral and low-expressing cells. The sigmoidal function ensures that rare positive events can still influence the overall mean. For negative markers, the sigmoidal curve is inverted to favor dimmer cells. The sigmoidal function can be expressed as:

$$Sigmoidal Function = \frac{1}{( 1 + {e}^{- 1} )}$$

The inflection point in the sigmoid curve can be calibrated using any of several techniques. For example, the inflection point can be fixed somewhere between [0,1] (e.g., two-thirds down the fluorescent intensity range). As another example, the inflection point can be aligned to a user-provided fluorescent intensity value. As yet another example, the inflection point can be aligned to an auto-gated fluorescent intensity value (e.g., gating on the right shoulder of the fluorescent intensity distribution curve).

At step 2904, the normalized intensity measurements from step 2902 are transformed using the calibrated sigmoidal function from step 2904. This yields sigmoidally-transformed intensity values (I″).

At step 2906, the system computes weights for each phenotype as the root product of the transformed intensity values (I″) of the phenotypes' component protein markers. Thus, for a single protein marker phenotype, the weight can be the computed value of I″ for that protein marker. For a phenotype defined by a combination of two protein markers, the weight can be computed as the square root of the product of the I″ values for the two component protein markers; for a phenotype defined by a combination of three protein markers, the weight can be computed as the cube root of the product of the I″ values for the three component protein markers, and so on for longer protein marker combinations. This operation ensures that rare double-positive cells are not drowned out by strong expression in one marker or the other. Step 2906 can produce a phenotype-specific weight for each cell in the single cell data 200.

At step 2908, the system computes the average cell weight for a subject phenotype within each sample. This can be computed as the average of all of the weights computed at step 2906 for the subject phenotype with respect to all of the cells of a given sample (and repeating this computation for all of the samples). Accordingly, step 2908 can produce a combinatoric matrix that is similar to matrix 920 of FIG. 9B except the cells of the matrix produced by step 2908 will be populated with the average cell weights for each defined patient-phenotype combination (whereas the cells of matrix 920 in FIG. 9B are populated with patient-specific phenotype frequencies). Step 2908 produces a threshold-free, unitless, patient-level measure of phenotype abundance that can be compared to a clinical variable like disease status or treatment outcome.

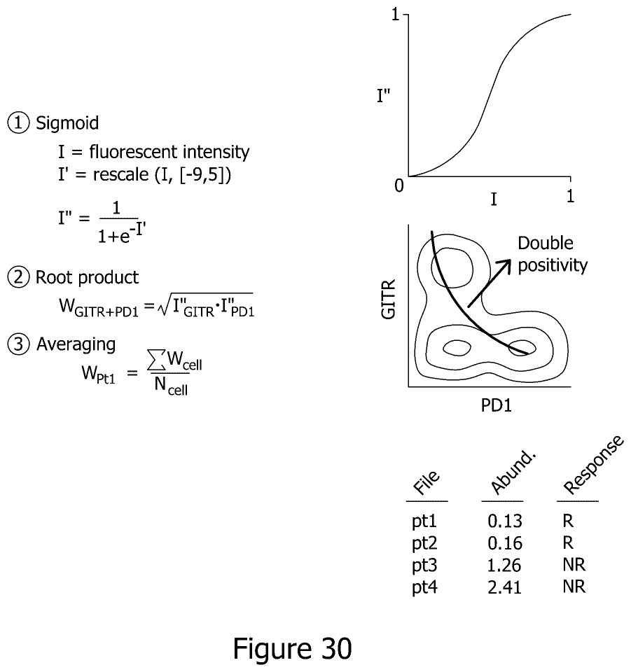

FIG. 30 depicts an example of how this non-gating approach can be applied to a given phenotype with two protein markers (GITR and PD1). The series of transformations shown by FIG. 30 operate together to inflate the expression of rare double-positive cells over single and double-negative cells.

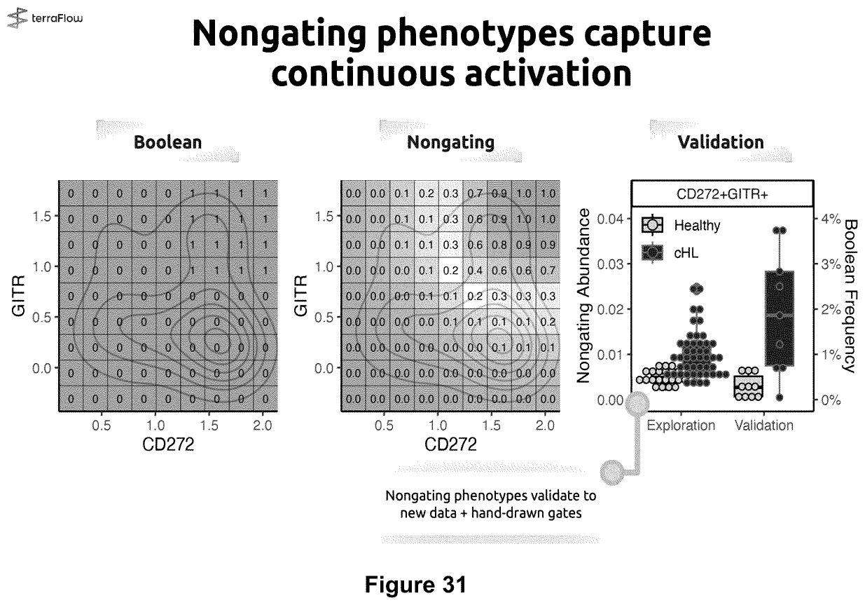

This non-gating approach effectively blurs the boundary between expressing and non-expressing cells. The brightest cells still have a value of 1 and the dimmest cells still have a value of 0, but now the intermediate cells take on a range of values based on their relative double-positivity (see, for example, FIG. 31)

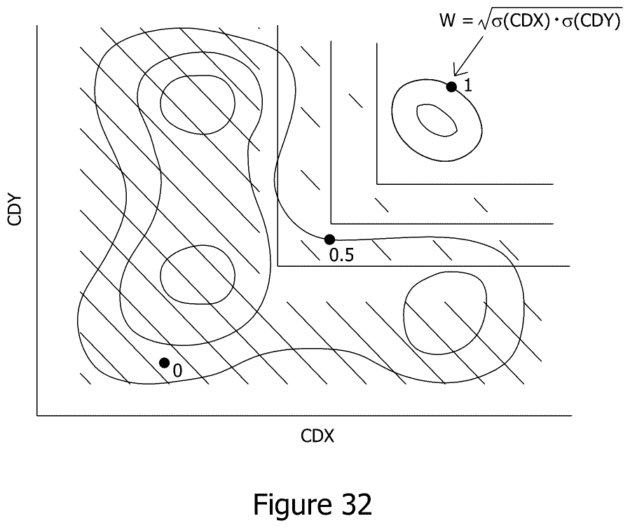

Accordingly, it should be understood that the non-gating approach can also be applied to complex phenotypes such as CD3+CD4+ because step 2906 takes the root product of the component markers: \sqrt{σ(I_{CD3})·σ(I_{CD4})} (see FIG. 32).

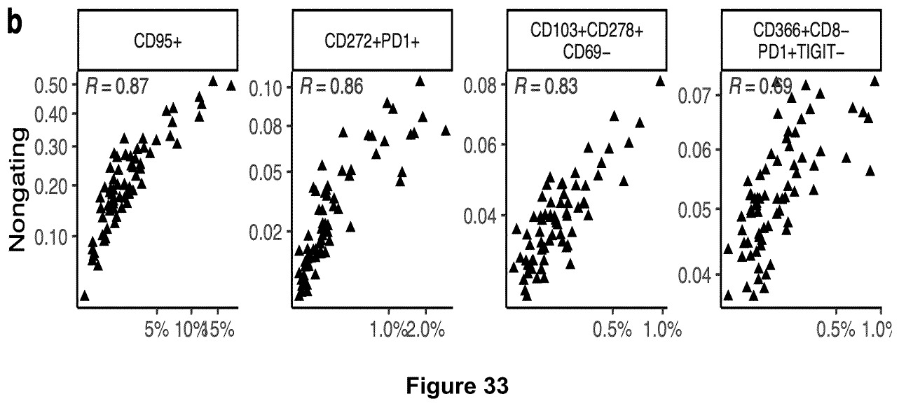

When the non-gating approach is applied to a real-life Hodgkin's dataset, the non-gating approach produces similar results (see FIG. 33) as would be produced via a gating approach. Here, we measured the abundance of four phenotypes using expert-set traditional gates and the innovative non-gating approach described herein. The same patients (triangles) show up as high expressors when using either method. In cases where the non-gating and traditional approaches diverge, the non-gating approach captures continuous modes of protein up-regulation that are lost using traditional gates.

Non-gating abundances can be interpreted directly or used to flag disease-associated phenotypes for further processing. For example, phenotypes flagged by the non-gating approach can be gated manually by a human reviewer. They can also be gated automatically using a foundation model pre-trained on existing gating strategies. This approach allows the system to quickly screen disease-associated populations without having to compute a separate gate for each phenotype.

While the invention has been described above in relation to its example embodiments, various modifications may be made thereto that still fall within the invention's scope.

For example, while the examples described above are focused on flow cytometry data, the techniques described herein can also be used for other types of single cell data such as data derived from mass cytometry, transcript expression (RNA-seq)-such as gene expressions using HGNC data, metabolite expression (mass spectrometry) such as Human Metabolome Database (HMDB) data, chromatin accessibility (ATAC-seq) such as ENCODE data, and multiomic techniques such as CITE-seq that combine two or more omic data types (e.g., HGNC+UniProt). In an example use case where ssRNAseq data is used, a practitioner can map molecular features to their HGNC gene IDs instead of their UniProt protein IDs. Moreover, the index process can be modified to the specific phraseology of RNA sequencing. For example, the extraction engine may look for keywords such as “highly expressed” instead of “bright”. Similarly, these techniques could also be used for use cases where there are multiomic data sets such as CITEseq where each cell type is defined by a combination of protein and gene IDs.

These and other modifications to the invention will be recognizable upon review of the teachings herein.

What is claimed is:

1. A multi-agent system for transforming single call data into one or more insights about one or more phenotypes exhibited by the single cell data, the system comprising:
one or more processors;
one or more databases configured to provide a plurality of linkages between (1) phenotype data for a plurality of phenotypes derived from the single cell data and (2) external knowledge about the phenotypes derived from medical literature;
one or more first agents for execution by the one or more processors, wherein the one or more first agents are configured to transform the single cell data into the phenotype data;
one or more second agents for execution by the one or more processors, wherein the one or more second agents are configured to process a corpus of medical literature using natural language processing (NLP) to determine information relating to the phenotypes described by the medical literature, wherein the external knowledge comprises the determined information; and
one or more third agents for execution by the one of more processors, wherein the one or more third agents are configured to synthesize one or more insights about the single cell data with respect to one or more phenotypes of interest, wherein the one or more insights are derived from the linkages in the one or more databases; and
wherein the first, second, and third agents are configured for operating in parallel with each other.

2. The system of claim 1 wherein the single cell data comprises data indicative of protein expression levels for a plurality of different protein markers in a plurality of different single cells with respect to a plurality of subjects, and wherein each subject is associated with status data for the defined outcome;
wherein each phenotype is defined by a protein marker combination of one or more of the protein markers in the single cell data;
wherein the phenotype data created by the one or more first agents comprises a first computer-readable data structure that defines a network model of related phenotypes, the network model comprising a plurality of connected nodes, each node corresponding to a different phenotype from among a plurality of the phenotypes derived from the single cell data and being associated with relationship data for its corresponding phenotype to the defined outcome, wherein the network model connects the nodes according to a network structure, and wherein the network structure defines connections within the network model between nodes where the nodes' corresponding phenotypes differ by the addition or removal of 1 protein marker;
wherein the determined information created by the one or more second agents comprises a second computer-readable data structure that defines an index of a plurality of medical papers that reference one or more of the phenotypes, wherein the medical papers are indexed according to the phenotypes referenced therein, and wherein the index also associates the medical papers with metadata about the medical papers;
wherein the one or more databases comprise the first computer-readable data structure and the second computer-readable data structure; and
wherein the one or more third agents are configured to access the first and second computer-readable data structures to (1) identify the one or more phenotypes of interest based on (i) the network structure of the network model and (ii) the relationship data of the network model and (2) synthesize the one or more insights about the single cell data with respect to the identified one or more phenotypes of interest, wherein the one or more insights are derived from one or more of the medical papers that are linked to the identified one or more phenotypes of interest indexed by the second computer-readable data structure.

3. The system of claim 2 wherein the third agent is further configured to traverse the first computer-readable data structure based on the network structure of the network model to find one or more of the nodes that qualify as local peaks with respect to the relationship data, wherein the one or more local peaks serve as the identified one or more phenotypes of interest.

4. The system of claim 2 wherein the third agent is further configured to traverse the first computer-readable data structure based on the network structure of the network model to determine a shortest phenotype whose relationship to the defined outcome satisfies defined criteria, wherein the identified one or more phenotypes of interest comprises the determined shortest phenotype whose relationship to the defined outcome satisfies the defined criteria.

5. The system of claim 2 wherein the third agent is further configured to (1) find a subset of the nodes that correspond to phenotypes whose relationship data exhibits a strong relationship to the defined outcome according to first defined criteria, (2) arrange the nodes in the subset into one or more clusters of nodes based on the network structure of the network model so that each cluster includes nodes of the subset that are directly connected according to the network structure, and (3) select one or more phenotypes of interest within each of the one or more clusters according to second defined criteria.

6. The system of claim 2 wherein the third agent is further configured to provide first data and second data to a natural language generation (NLG) platform to cause the NLG platform to produce a natural language output that serves as the one or more insights;
wherein the first data is representative of the identified one or more phenotypes of interest;
wherein the second data is representative of metadata about the one or more medical papers that are linked by the second computer-readable data structure to the one or more phenotypes of interest; and
wherein the natural language output summarizes the linked one or more medical papers with respect to the identified one or more phenotypes of interest.

7. The system of claim 2 wherein the one or more second agents or the one or more third agents are configured to score a plurality of the medical papers to quantify strengths of their relationships to the phenotypes that they reference according to defined criteria, and wherein the one or more third agents are further configured to select which of the linked one or more medical papers to use for synthesizing the insight based on the scores for the linked one or more medical papers.

8. The system of claim 7 wherein the one or more second or third agents are further configured to compute notability scores for a plurality of the medical papers and/or relevance scores for a plurality of the medical papers.

9. The system of claim 8 wherein the one or more second or third agents are further to compute the notability scores based on which of a plurality of sections of the medical papers reference a subject phenotype.

10. The system of claim 8 wherein the one or more second or third agents are further configured to compute the relevance scores based on relevances of the medical papers to a defined topic of interest.

11. The system of claim 7 wherein the one or more second or third agents are further configured to compute a specificity score for a subject phenotype based on a quantification of how specific the subject phenotype is to medical papers addressing the subject phenotype and a defined topic of interest relative to other medical papers that address the subject phenotype but do not address the defined topic of interest.

12. The system of claim 7 wherein the one or more second or third agents are further configured to score a plurality of phenotypes based on an aggregation of notability criteria, relevance criteria, and specificity criteria.

13. The system of claim 2 wherein the one or more first agents are further configure to compute a plurality of correlation values for a plurality of the nodes of the network model based on how strongly the single cell data exhibits a correlation between the nodes' corresponding phenotypes and the defined outcome, wherein the relationship data comprises the correlation values.

14. The system of claim 13 wherein the one or more first agents are further configured to weight the correlation scores according to a weighting that favors shorter phenotypes over longer phenotypes.

15. The system of claim 1 wherein the one or more third agents comprise a plurality of different third agents that are configured to perform different synthesis operations.

16. The system of claim 1 wherein the one or more second agents are configured to perform NLP on the corpus of medical literature to (1) identify medical papers that reference phenotypes, (2) determine the phenotypes that are referenced by the identified medical papers, (3) determine metadata for the identified medical papers, and (4) create a data structure so that the identified medical papers are associated with their determined phenotypes and metadata.

17. A multi-agent system for transforming single call data into one or more insights about one or more phenotypes exhibited by the single cell data, wherein the single cell data comprises data indicative of protein expression levels for a plurality of different protein markers in a plurality of different single cells with respect to a plurality of subjects, and wherein each subject is associated with status data for the defined outcome, the system comprising:
one or more processors; and
one or more memories configured to store a computer-readable data structure;
wherein the computer-readable data structure defines a network model of related phenotypes, the network model comprising a plurality of connected nodes, each node corresponding to a different phenotype from among a plurality of phenotypes derived from the single cell data and being associated with (1) relationship data for its corresponding phenotype to the defined outcome and (2) metadata about one or more medical papers that reference the corresponding phenotype;
wherein the one or more processors, in cooperation with the one or more memories, are configured to execute one or more first agents, one or more second agents, and one or more third agents;
wherein the one or more first agents are configured to build a portion of the computer-readable data structure through transformation of the single cell data;
wherein the one or more second agents are configured to (1) mine a corpus of the medical papers using natural language processing (NLP) to determine which of the medical papers qualify as phenotype-related medical papers that reference phenotypes and (2) link the phenotype-related medical papers with nodes of the network model by augmenting the nodes with associations to metadata about the phenotype-related medical papers based on correspondence between the nodes' corresponding phenotypes and the phenotypes referenced by the phenotype-related medical papers;
wherein the one or more third agents are configured to access the computer-readable data structure to synthesize an insight about the single cell data with respect to one or more phenotypes of interest, wherein the insight is derived from medical paper metadata associated by the computer-readable data structure with the one or more phenotypes of interest; and
wherein the one or more first, second, and third agents are configured for operating in parallel with each other.

18. The system of claim 17 wherein each phenotype is defined by a protein marker combination of one or more of the protein markers in the single cell data, wherein the network model connects the nodes according to a network structure, wherein the network structure defines connections within the network model between nodes where the nodes' corresponding phenotypes differ by the addition or removal of 1 protein marker; and
wherein the one or more third agents are further configured to identify the one or more phenotypes of interest based on (1) the network structure of the network model and (2) the relationship data of the network model.

19. The system of claim 18 wherein the one or more third agents are further configured to identify the one or more phenotypes of interest based on (1) the network structure of the network model, (2) the relationship data of the network model, and (3) the medical paper metadata.

20. An article of manufacture comprising:
processor-executable code resident on a non-transitory computer-readable storage medium, wherein the processor-executable code is configured for execution by one or more processors to cause the one or more processors to:
traverse a network model of related phenotypes that is derived from single cell data to identify one or more phenotypes of interest, wherein the network model comprises a plurality of nodes that are connected according to a network structure, wherein the nodes correspond to different phenotypes derived from the single cell data and are associated with relationship data between the nodes' corresponding phenotypes and a defined outcome according to the single cell data, wherein the phenotypes are defined by protein marker combinations of one or more protein markers from the single cell data, wherein the network structure defines connections within the network model between nodes where the nodes' corresponding phenotypes differ by the addition or removal of 1 protein marker, and wherein the one or more phenotypes of interest are identified based on (1) the network structure and (2) the relationship data;
determine medical literature metadata that is linked to the identified one or more phenotypes of interest and has been derived from a plurality of phenotype-related medical papers; and
synthesize an insight about the single cell data with respect to the identified one or more phenotypes of interest based on the determined medical literature metadata.
